(19) United States
(12) Patent Application Publication
(10) Pub. No.: US 2025/0085285 A1
(43) Pub. Date: Mar. 13, 2025

(54) Methods for Generating Biologically Specific Chromatin Condensates and Uses Thereof

(71) Applicant: The Regents of the University of California, Oakland, CA (US)

(72) Inventors: Muryam Gourdet, San Francisco, CA (US); Laura Hsieh, San Francisco, CA (US); Tracy Lou, San Francisco, CA (US); Geeta Narlikar, San Francisco, CA (US); Daniel Elnatan, San Francisco, CA (US)

(21) Appl. No.: 18/728,827

(22) PCT Filed: Jan. 18, 2023
(86) PCT No.: PCT/US2023/011055

§ 371 Date: Jul. 12, 2024

Related U.S. Application Data
(60) Provisional application No. 63/300,532, filed on Jan. 18, 2022.

(51) Int. Cl. G01N 33/574; G01N 33/58

(52) U.S. Cl. CPC G01N 33/57484; G01N 33/582; G01N 2500/20

(57) ABSTRACT

Compositions and methods for phase-separation of chromatin in distinct states are provided. In particular, differentially decorated chromatin in different states can be isolated by phase separation according to the methods disclosed herein. In some cases, specific chromatin interacting proteins are added to chromatin to recreate specific, biologically relevant chromatin states in vitro, which can be phase-separated using the disclosed methods. In addition, methods of using phase-separated chromatin for in vitro screening of pharmacological agents that modulate chromatin states, including small molecule chemicals and biomolecules, are also provided.

### CROSS-REFERENCE TO RELATED APPLICATIONS

This application claims the benefit of U.S. Provisional Patent Application No. 63/300,532, filed Jan. 18, 2022, which application is incorporated herein by reference in its entirety.

### STATEMENT REGARDING FEDERALLY SPONSORED RESEARCH OR DEVELOPMENT

This invention was made with government support under Grant No. R35 GM127020 and Grant No. U01 DK127421 awarded by the National Institutes of Health and Grant No. 1921794 awarded by the National Science Foundation. The government has certain rights in the invention.

### BACKGROUND OF THE INVENTION

Many diseases, including cancer, neurodegenerative diseases, and autoimmune diseases are driven by global changes in the organization and structure of chromatin. It is becoming increasingly evident that chromatin regulation is central to disease development and progression. Two current avenues where therapeutics are being developed for manipulating chromatin are: small molecule drugs and stem cell therapies. However, small molecule drugs targeting single genes have produced insufficient results for treating diseases. This is because i) many diseases are a result of multiple mutations, leading to global genome dysregulation, and ii) current small molecule drugs target proteins that are involved both in cancer progression and normal cellular processes, which lead to unwanted side effects. Additionally, stem cell therapies have been limited by inefficiencies in generating induced pluripotent stem (iPS) cells and in directing the differentiation pathways of stem cells because certain chromatin states that inhibit generation of iPS cells are difficult to disrupt. Thus, large disease markets are still awaiting effective treatments.

There remains a need for better methods of targeting and manipulating chromatin states to control cellular function and for screening for molecules that manipulate chromatin states, particularly disease-specific and stem cell-specific chromatin states. However, screening for small molecule drugs in disease through single protein or single pathway targeting has proved nearly impossible.

### SUMMARY OF THE INVENTION

Compositions and methods for phase-separation of chromatin in distinct states are provided. In particular, differentially decorated chromatin in different states can be isolated by phase separation according to the methods disclosed herein. In some cases, specific chromatin interacting proteins are added to chromatin to recreate specific, biologically relevant chromatin states in vitro, which can be phase-separated using the disclosed methods. In addition, methods of using phase-separated chromatin for in vitro screening of pharmacological agents that modulate chromatin states, including small molecule chemicals and biomolecules, are also provided.

In one aspect, a method of assessing a test agent for the ability to modulate a chromatin state of interest is provided, the method comprising: contacting a phase-separated droplet comprising a nucleosome array condensate with the test agent; and assessing the nucleosome array condensate for a modulation of the chromatin state of interest by the test compound. In some embodiments, provided are methods of assessing a test agent for the ability to target and modulate a specific and whole chromatin state of interest through disruption of multivalent interactions rather than targeting a single protein.

In certain embodiments, the test agent is a small molecule.

In certain embodiments, the test agent is a biomolecule. In some embodiments, the biomolecule is a polypeptide.

In certain embodiments, the chromatin state of interest is a naturally occurring chromatin state. In some embodiments, the naturally occurring chromatin state is a chromatin state of a normal cell. In other embodiments, the naturally occurring chromatin state is a chromatin state associated with a disease. In some embodiments, the disease is cancer (e.g., the chromatin state may be specific to a type of cancer). In some embodiments, the disease is a neurodegenerative disease (e.g., the chromatin state may be specific to a type of neurodegenerative disease). In some embodiments, the disease is an autoimmune disease (e.g., the chromatin state may be specific to a type of autoimmune disease).

In certain embodiments, the nucleosome array condensate comprises histone modifications. For example, the nucleosome array condensate may comprise one or more histone modifications selected from H3K9me3, H4K20me3, H3K27me3, H3K4me3, H3K36me3, H3K79me3, H3K56ac, H3K9Ac, and H4K16Ac, or any combination thereof. In some embodiments, the one or more histone modifications have a nucleosome occupancy selected to mimic a naturally occurring chromatin state. In some embodiments, the nucleosome occupancy is 60% or greater.

In certain embodiments, the nucleosome array condensate comprises one or more histone variants. In some embodiments, the one or more histone variants comprise H2Az, macro H2A, H3.3, CENP-A, H2ABBD, or any combination thereof. In some embodiments, the one or more histone variants are selected to mimic a naturally occurring chromatin state.

In certain embodiments, the nucleosome array condensate comprises one or more heterochromatin proteins bound to the one or more histone modifications. In some embodiments, the one or more heterochromatin proteins comprise chromobox protein homolog 5 (CBX5), chromobox protein homolog 3 (CBX3), chromobox protein homolog 1 (CBX1), or any combination thereof.

In certain embodiments, the one or more heterochromatin proteins exhibit a specificity for histones of the nucleosome array of 2-fold or greater as compared to the specificity for histones of a corresponding nucleosome array in which the one or more histone modifications are absent.

In certain embodiments, the one or more heterochromatin proteins exhibit a dissociation constant (K_{D}) for the nucleosome array of less than or equal to 5 μM.

In certain embodiments, the nucleosome array condensate comprises one or more proteins that associate with chromatin or DNA. In some embodiments, the one or more proteins that associate with chromatin or DNA comprise a polycomb-group protein such as, but not limited to, PRC1 and PRC2; a bromodomain-containing protein such as, but not limited to BRD1, BRD2, BRD3, BRD4, BRD5, BRD6, BRD7, BRD8, or BRD9; a chromobox protein homolog such as, but not limited to CBX1, CBX2, CBX3, CBX4, CBX5, CBX6, CBX7, or CBX8; a mediator complex subunit such as, but not limited to, head module subunits: MED6, MED8, MED11, SRB4/MED17, SRB5/MED18, ROX3/MED19, SRB2/MED20 and SRB6/MED22, middle module subunits: MED1, MED4, NUT1/MED5, MED7, CSE2/MED9, NUT2/MED10, SRB7/MED21 and SOH1/MED31. CSE2/MED9, tail module subunits: MED2, PGD1/MED3, RGR1/MED14, GAL11/MED15 and SIN4/MED16, and CDK8 module subunits: MED12, MED13, CCNC and CDK8; or an oncogenic fusion protein (e.g., that acts as a dysregulated transcription factor or as a chromatin regulator, or interacts with chromatin-modifying enzymes or epigenetic complexes that modulate chromatin); or any combination thereof.

In certain embodiments, the one or more heterochromatin proteins are labeled, and wherein assessing the nucleosome array condensate for a modulation of the chromatin state of interest by the test compound comprises detecting the labeled heterochromatin proteins.

In certain embodiments, the nucleosome array condensate comprises labeled DNA, wherein assessing the nucleosome array condensate for a modulation of the chromatin state of interest by the test compound comprises detecting the labeled DNA.

In another aspect, a pharmaceutical composition comprising a test agent, identified as modulating a chromatin state of interest by a method described herein, is provided.

In another aspect, a method comprising administering to an individual in need thereof an effective amount of a test agent, identified as modulating a chromatin state of interest by a method herein, is provided.

In another aspect, a method of producing a nucleosome array condensate that mimics a naturally occurring chromatin state is provided, the method comprising: combining one or more heterochromatin proteins and a nucleosome array under conditions in which the nucleosome array forms a nucleosome array condensate that mimics a naturally occurring chromatin state, wherein the nucleosome array comprises histone modifications, histone variants, or both, selected to mimic the naturally occurring chromatin state.

In certain embodiments, the heterochromatin protein comprises CBX5, CBX3, CBX1, or any combination thereof.

In certain embodiments, the one or more heterochromatin proteins exhibit a specificity for histones of the nucleosome array of 2-fold or greater as compared to the specificity for histones of a corresponding nucleosome array in which the one or more histone modifications and/or histone variants are absent.

In certain embodiments, the one or more heterochromatin proteins exhibit a dissociation constant (K_{D}) for the nucleosome array of less than or equal to 5 μM.

In certain embodiments, the one or more heterochromatin proteins are labeled.

In certain embodiments, the nucleosome array comprises histone modifications comprising H3K9me3, H4K20me3, H3K27me3, H3K4me3, H3K36me3, H3K79me3, H3K56ac, H3K9Ac, H4K16Ac, or any combination thereof, selected to mimic the naturally occurring chromatin state.

In certain embodiments, the nucleosome array comprises histone variants comprising H2Az, macro H2A, H3.3, CENP-A, H2ABBD, or any combination thereof, selected to mimic the naturally occurring chromatin state.

In certain embodiments, the nucleosome array comprises labeled DNA.

In another aspect, a method to distinguish nucleosome array condensates that have different chromatin states is provided, the method comprising: a) performing fluorescence imaging of fluorescently labeled nucleosome array condensates to obtain fluorescence images; b) performing segmentation-free image quantification on the fluorescence images; c) computing size distributions of the nucleosome array condensates from the fluorescence images; d) reducing complexities of the size distributions into distinct features that discriminate between the fluorescence images to simplify quantification of the nucleosome array condensates; e) acquiring shape distributions for the nucleosome array condensates from the fluorescence images; and f) reducing complexities of the shape distributions into distinct features that discriminate between the fluorescence images to simplify quantification of the nucleosome array condensates. In some embodiments, the method further comprises quantifying z-factors using the outputs from steps d) and f) to assess quality control and reproducibility. In some embodiments, the method further comprises comparing data from multiple screens to identify test agents that specifically modulate the chromatin state of interest.

In another aspect, a computer implemented method for processing fluorescence images of fluorescently labeled nucleosome array condensates is provided, the computer performing steps comprising: a) receiving the fluorescence images of the fluorescently labeled nucleosome array condensates; b) performing segmentation-free image quantification on the fluorescence images; c) computing size distributions of the nucleosome array condensates from the fluorescence images; d) reducing complexities of the size distributions into distinct features that discriminate between the fluorescence images to simplify quantification of the nucleosome array condensates; e) acquiring shape distributions for the nucleosome array condensates from the fluorescence images; and f) reducing complexities of the shape distributions into distinct features that discriminate between the fluorescence images to simplify quantification of the nucleosome array condensates. In some embodiments, the method further comprises quantifying z-factors using the outputs from steps d) and f) to assess quality control and reproducibility. In some embodiments, the method further comprises comparing data from multiple screens to identify test agents that specifically modulate the chromatin state of interest.

In another aspect, a non-transitory computer-readable medium comprising program instructions that, when executed by a processor in a computer, causes the processor to perform the computer implemented method described herein is provided.

In another aspect, a system for distinguishing nucleosome array condensates that have different chromatin states is provided, the system comprising: a processor programmed to process fluorescence images of fluorescently labeled nucleosome array condensates according to the computer implemented method described herein; and a display component for displaying information about the nucleosome array condensates regarding size distributions of the nucleosome array condensates, shape distributions of the nucleosome array condensates, quantification of the nucleosome array condensates, or histone modifications or DNA modifications, or any combination thereof.

In certain embodiments, the system further comprises reagents for forming a phase-separated droplet comprising a fluorescently labeled nucleosome array condensate.

In certain embodiments, the system further comprises a test agent.

### BRIEF DESCRIPTION OF THE DRAWINGS

FIG. 1. Schematic of H3K9me3 F40 sortase mediated ligation chemistry and purification. A) A schematic of the semi-synthesis of histone H3 modifications via F40 sortase mediated chemical ligation. Purified truncated histone H3 (H3Δ32), H3K9me3 depsipeptide, and F40 sortase are added together to a reaction to synthesize site-specific modified histone H3. B) Further purification of ligated histone H3 by cation-anion column purification using HiTrap Q and SP HP. C) A schematic demonstrating using purified ligated histone H3 for creating site-specific histone modified octamer.

FIGS. 2A-2B. Confirmation of successful ligation by Western blot and liquid chromatography-mass spectrometry (LC-MS). FIG. 2A shows a Western blot. FIG. 2B shows LC-MS with calculated masses for H3C110A and H3K9me3. There is a 43 Dalton difference in mass between wild-type (WT) H3C110A and H3K9me3 as is expected for trimethylation.

FIGS. 3A-3C. Model of HP1 and chromatin organization to induce phase separation. FIG. 3A shows a diagram of HP1α and its domains, of Cy3 labeled HP1αKCK, and of AlexaFluor-647 labeled 12N H3K9me3 chromatin array. FIG. 3B shows a schematic demonstration of HP1α assembling onto chromatin and forming phase separated droplets. FIG. 3C shows a series of brightfield and fluorescent images showing HP1α-chromatin condensates in vitro.

FIG. 4. H3K9me3 chromatin specificity over WT chromatin. The figure shows there is biological specificity for H3K9me3 modified chromatin by HP1α. Unmodified WT chromatin requires 3-fold higher concentration of HP1α to achieve equivalent phasing abilities. The images show a titration of HP1α with 40 nM unmodified and 40 nM modified chromatin.

FIG. 5. Schematic of assay for HP1-chromatin condensate screening. The figure shows a schematic of how to utilize the HP1α-chromatin platform in a high-throughput screening method. Phase separated droplets are formed and then dispensed using an acoustic liquid dispenser (Biomek FX) into 384 well plates with small molecule libraries. These droplets are rapidly imaged using a high-content imaging microscope (INCell Analyzer).

FIG. 6. Effects of Sgo1 and HP1β on HP1-chromatin condensates. The figure shows HP1α-chromatin condensates formation affected by biologically relevant interaction partners. Sgo1 peptide (derived from the Shugoshin protein) enhances condensates by making them larger. HP1β dissolves condensates, making them disappear or smaller.

FIG. 7. Effects of bioactive small molecules on HP1-chromatin condensates. The figure shows HP1α-chromatin condensates affected by bioactive small molecules. Vemurafenib and Lapatinib ditosylate show mild or no effect on condensates. Erlotinib and Vismodegib enhance condensate formation. Zileuton and Amonifide dissolve condensates.

FIG. 8. Array DNA column purification and fluorescent labeling. The figure shows a schematic diagram of chromatin array DNA purification and confirmation by acrylamide gel. Large amounts of plasmid are grown in E. coli and purified by QIAGEN Gigaprep kits. This DNA is then linearized by restriction enzymes EcoRV and XhoI. The DNA is further column purified with a custom packed Sephacryl 5-1000 SF gel filtration column. Purified array DNA is then fluorescently labeled with AlexaFluor-647 with Klenow fragment. This fluorescently labeled DNA is used with octamer to assemble nucleosomal arrays.

FIG. 9. Results of screening for small molecule modulators of phase separated HP1 chromatin droplets. The library of small molecules consists of bioactives. The table shows the number and percentage of small molecules within the library that inhibited droplet formation, promoted droplet formation, did not affect the droplets or affected the droplets in an alternative way.

FIG. 10. Area opening of a fluorescence image. A line profile (orange) of image intensities illustrates the result of opening an image with increasing area thresholds. The black line shows a line profile of the original image with intact peaks. After opening with an area threshold of 628 pixels-squared (equivalent to a circle with radius of 10 pixels), some of the intensities from the peaks have been removed to give a surface area equal to the threshold. This process produces flat peaks (orange, right). Image opening with larger area threshold (purple), equivalent to a circle with radius of 15 pixels, shows further peak height reduction and larger surface areas.

FIG. 11. Size distributions from fluorescence images of condensates. Size distributions computed from a few examples of different condensate morphologies are also distinct. These distributions capture visual qualities within the image which can be used to discriminate the condensates. Variations between distributions from the same sample from 5 different fields reflect the heterogeneities in the actual sample and may be used as a reproducibility metric.

FIG. 12. Distribution-derived moments quantifies differences and similarities between images. Points with the same color and plotting point styles are computed from size distributions of different fields of the same sample from FIG. 10. Shown here are the first moment (mean) on the x-axis versus second moment (variance) on the y-axis. These points cluster near one another when their size distributions are also similar in shape.

FIG. 13. Size distribution overlay of single images from each well. Intensity scaling on each image is arbitrary.

FIG. 14. Scatter Plots of the moments derived from the distributions.

FIG. 15. Heterochromatin and euchromatin droplets do not readily mix. Fluorescently labeled HP1 heterochromatin droplets with H3K9me3 are shown in the upper left panel. Fluorescently labeled Brd4 euchromatin droplets with H3K27ac are shown in the upper right panel. As shown in the merged image (lower panel), the different types of chromatin condensates do not readily mix.

FIG. 16. H3.3K27M mutation affects PRC2 chromatin condensate formation. Human PRC2 complex was added to both WT H3.3K27me3 nucleosome arrays and H3.3K27M nucleosome arrays. Droplets are visualized with end-labeled DNA.

### DETAILED DESCRIPTION

Compositions and methods for phase-separation of chromatin in distinct states are provided. In particular, differentially decorated chromatin in different states can be isolated by phase separation according to the methods disclosed herein. In some cases, specific chromatin interacting proteins are added to chromatin to recreate specific, biologically relevant chromatin states in vitro, which can be phase-separated using the disclosed methods. In addition, methods of using phase-separated chromatin for in vitro screening of pharmacological agents that modulate chromatin states, including small molecule chemicals and biomolecules, are also provided.

Before the present compositions comprising phase-separated chromatin and methods of making and using them for in vitro screening of pharmacological agents that modulate chromatin states are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a histone” includes a plurality of such histones and reference to “the nucleosome” includes reference to one or more nucleosomes and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

By “isolated” is meant, when referring to a nucleic acid or a protein, that the indicated molecule is separate and discrete from the whole organism with which the molecule is found in nature or is present in the substantial absence of other biological macro molecules of the same type.

“Substantially purified” generally refers to isolation of a substance (compound, nucleic acid, or protein) such that the substance comprises the majority percent of the sample in which it resides. Typically in a sample, a substantially purified component comprises at least 50%, preferably 80%-85%, or more preferably 90-95% of the sample. Techniques for purifying nucleic acids or proteins of interest are well-known in the art and include, for example, ion-exchange chromatography, affinity chromatography, gel filtration, and sedimentation according to density.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide.

As used herein, the term “biological sample” includes any cell or tissue or bodily fluid containing chromatin from a eukaryotic organism, such as cells from plants, animals, fungi, or protists. The biological sample may include cells from a tissue or bodily fluid, including but not limited to, blood, saliva, cells from buccal swabbing, fecal matter, urine, bone marrow, spinal fluid, lymph fluid, skin, organs, and biopsies, as well as in vitro cell culture constituents, including recombinant cells and tissues grown in culture medium.

### Nucleosome Array Condensates

The subject methods use phase separation to isolate chromatin in specific states. Multiple natural chromatin states that are present in cells can be simulated through the customization of chromatin differentially decorated with specific chromatin interacting proteins and comprising various histone and DNA modifications. The disclosed methods allow recreation of biologically relevant chromatin states in phase-separated nucleosome array condensates that can be analyzed in vitro.

The nucleosome represents a basic structural unit of chromatin. A nucleosome consists of 147 base pairs of DNA wrapped approximately 1.7 times around an octamer of histone proteins. The histone octamer is composed of four homodimers of the histone core proteins: H2A, H2B, H3 and H4. The histone core proteins are positively charged and bind to anionic DNA through strong electrostatic interactions.

The nucleosome arrays are synthetic (i.e., not obtained from naturally existing chromatin) and can be constructed in vitro from DNA molecules comprising a series of nucleosome positioning sequences. Natural nucleosome positioning sequences typically contain ten base pair repeats of a TA dinucleotide sequence. In some embodiments, nucleosome arrays are constructed with artificial nucleosome positioning sequences that have higher histone affinities than natural positioning sequences. For example, a nucleosome array can be constructed using the ‘601’ nucleosome positioning sequence, which has a high histone affinity (For a description of the ‘601’ nucleosome positioning sequence, see, e.g., Lowary & Widom (1998) New DNA sequence rules for high affinity binding to histone octamer and sequence-directed nucleosome positioning, J. Mol. Biol. 276, 19-42; herein incorporated by reference in its entirety). DNA constructs containing an array of defined nucleosome positioning sites can be generated using Gibson Assembly cloning (see, e.g., Gibson et al. (2019) Organization of chromatin by intrinsic and regulated phase separation, Cell 179, 470-484; herein incorporated by reference in its entirety). See Examples for a description of a method of generating a nucleosome array using a plasmid containing 12×601 array DNA and its propagation in a suitable cell line.

The array DNA can be cut from a plasmid, for example, using restriction enzymes. Typically, a rare cutter such as a 6-base cutter is used. Exemplary restriction enzymes that recognize 6 bp sequences of DNA (i.e., 6-base cutters) include, but are not limited to, AclI, HindIII, SspI, BspLU11I, AgeI, MluI, SpeI, BglII, Eco47III, StuI, ScaI, ClaI, AvaIII, VspI, MfeI, PmaCI, PvuII, NdeI, NcoI, SmaI, SadI, AvrII, PvuI, XmaIII, SplI, XhoI, PstI, AflII, EcoRI, AatII, Sad, EcoRV, SphI, Nad, BsePI, NheI, BamHI, NarI, ApaI, KpnI, SnaI, SalI, ApaLI, HpaI, SnaBI, BspHI, BspMII, NruI, XbaI, BclI, MstI, BalI, Bsp1407I, PsiI, AsuII, and AhaIII. In some embodiments, array DNA is cut with a restriction enzyme that creates a 5′ overhang (e.g., XhoI) to facilitate incorporation of a fluorescent nucleotide label, for example, with Klenow fragment.

Before addition of histones, the array DNA may be isolated and further purified. Methods for isolating and purifying DNA are well known in the art. See, e.g., Green & Sambrook Molecular Cloning: A Laboratory Manual (Cold Spring Harbor Laboratory Press; 4^{th} edition, 2012; herein incorporated by reference). For example, the array DNA can be isolated and purified by phenol-chloroform extraction, ethanol precipitation, and gel filtration (see Examples).

Histones are added to the array DNA to produce the nucleosome array. Nucleosome arrays can be assembled using salt gradient dialysis (see, e.g., Sanulli et al. (2019) Nature 575:390-394; herein incorporated by reference in its entirety). In addition, non-histone proteins may be added to the nucleosome array to simulate a naturally occurring chromatin state. Non-histone proteins may include, without limitation, heterochromatin proteins (e.g., chromobox protein homolog (CBX) proteins), scaffold proteins, Polycomb, DNA-modifying proteins, DNA-binding proteins, histone-modifying enzymes, histone-binding proteins, and the like.

Phase-separated droplets containing the nucleosome array are formed by contacting the nucleosome array with a DNA-binding protein such as HP1α that promotes DNA compaction and phase-separation. See, e.g., Examples for a description of methods of producing nucleosome array condensates using HP1α. Similarly, phase-separated droplets containing the nucleosome array may be formed by contacting the nucleosome array with a protein such as PRC2. See, e.g., Examples for a description of methods of producing nucleosome array condensates using PRC2.

In certain embodiments, the nucleosome array condensate comprises histone modifications selected to mimic a naturally occurring chromatin state. For example, the nucleosome array condensate may comprise one or more histone modifications, including, methylation, acetylation, citrullination, phosphorylation, SUMOylation, ubiquitination, and/or ADP-ribosylation. Exemplary histone modifications include, without limitation, H3K9me3, H4K20me3, H3K27me3, H3K4me3, H3K36me3, H3K79me3, H3K56ac, H3K9Ac, and H4K16Ac.

In some embodiments, one or more histone modifications in a nucleosome array have a nucleosome occupancy selected to mimic a naturally occurring chromatin state. In certain embodiments, the nucleosome occupancy is at least 40%, at least 50%, at least 60%, at least 70%, at least 80%, at least 90%, or greater. In certain embodiments, the nucleosome occupancy is 40% to 100%, including any percentage nucleosome occupancy in this range such as 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100%.

In certain embodiments, the nucleosome array condensate comprises one or more histone variants, which may be selected to mimic a naturally occurring chromatin state. For example, the nucleosome array may comprise one or more histone variants of H2A, H2B, H3, and/or H4. Exemplary histone variants include, without limitation, H2Az, macro H2A, H3.3, CENP-A, H2ABBD, H3.1, H3.2, TS H3.4, H4.V, H2A.X, H2A.Z.2, H2A.B, H2A.L, H2A.P, H2A.J, H2B.1, H2B.W, H2B.Z, and H2B.E. Additional histone variants are listed in the National Center for Biotechnology histone database “HistoneDB 2.0—with variants” (see ncbi.nlm.nih.gov/research/HistoneDB2.0/).

As surprisingly demonstrated herein, a nucleosome array condensate may be designed to mimic a naturally occurring (e.g., pathological or non-pathological) chromatin state by selecting the ratio of octamers comprising a particular histone modification and/or histone variant for incorporation into the nucleosome array, where the selected ratio of octamers comprising a particular histone modification and/or histone variant is based on a known ratio of octamers comprising a particular histone modification and/or histone variant present in a particular naturally occurring chromatin state of interest—non-limiting examples of which include chromatin states found in cells of cancer types of interest, etc. As proof of principle, Example 4 herein demonstrates that the desired amount of a modified (H3K27me3) octamer and the amount of a mutant (H3K27M) octamer can be titrated into the assembly to quantitatively incorporate the desired octamer into the array. For example, in FIG. 16, 10% of the H3K27M mutation was added in the chromatin (that is, 10% of the octamers were comprised of H3K27M histones), which reflects the amount of H3K27M incorporated into the genome of diffuse intrinsic pontine glioma (DIPG) patients, where the resulting chromatin state was determined to mimic the naturally occurring chromatin state of DIPG cells. According to the methods of the present disclosure, such condensates mimicking pathological chromatin states may be used to screen for drugs (e.g., small molecules) that convert the pathological chromatin state to a non-pathological chromatin state. Drugs exhibiting this feature are identified as candidate drugs for treating the condition (e.g., cancer) with which the pathological chromatin state is associated.

In certain embodiments, the nucleosome array condensate comprises one or more heterochromatin proteins bound to the one or more histone modifications. Exemplary heterochromatin proteins include, without limitation, chromobox protein homolog 5 (CBX5), chromobox protein homolog 3 (CBX3), and chromobox protein homolog 1 (CBX1).

In certain embodiments, the one or more heterochromatin proteins exhibit a specificity for histones of the nucleosome array of 2-fold or greater as compared to the specificity for histones of a corresponding nucleosome array in which the one or more histone modifications are absent. In some embodiments, the one or more heterochromatin proteins exhibit a dissociation constant (K_{D}) for the nucleosome array of less than or equal to 5 μM.

In certain embodiments, the nucleosome array condensate comprises one or more proteins that associate with chromatin or DNA. In some embodiments, one or more proteins that associate with chromatin or DNA comprise a polycomb-group protein such as, but not limited to, PRC1 and PRC2; a bromodomain-containing protein such as, but not limited to BRD1, BRD2, BRD3, BRD4, BRD5, BRD6, BRD7, BRD8, and BRD9; a chromobox protein homolog such as, but not limited to CBX1, CBX2, CBX3, CBX4, CBX5, CBX6, CBX7, and CBX8; a mediator complex subunit such as, but not limited to, head module subunits: MED6, MED8, MED11, SRB4/MED17, SRB5/MED18, ROX3/MED19, SRB2/MED20 and SRB6/MED22, middle module subunits: MED1, MED4, NUT1/MED5, MED7, CSE2/MED9, NUT2/MED10, SRB7/MED21 and SOH1/MED31. CSE2/MED9, tail module subunits: MED2, PGD1/MED3, RGR1/MED14, GAL11/MED15 and SIN4/MED16, and CDK8 module subunits: MED12, MED13, CCNC and CDK8; or an oncogenic fusion protein (e.g., that acts as a dysregulated transcription factor or as a chromatin regulator, or interacts with chromatin-modifying enzymes or epigenetic complexes that modulate chromatin); or any combination thereof.

### Screening

Methods of screening for agents in vitro that modulate nucleosome array condensates are also provided. Nucleosome array condensates, prepared as described herein, may be analyzed to determine their responses to exposure to a test agent. Candidate test agents may include chromatin-modifying drugs, small molecules, and macromolecules, including DNA and histone-modifying molecules and compounds that regulate the activity of histone-modifying and DNA-modifying enzymes.

In some embodiments, a nucleosome array condensate, as described herein, is used as a disease model to determine the effects of a candidate agent on a disease associated-chromatin state. For example, chromatin from certain diseased cells or tissues may have an altered chromatin structure relative to the chromatin from normal or healthy cells and show epigenetic changes associated with disease progression. Therefore, analysis of a nucleosome array condensate mimicking the chromatin structure from such diseased cells or tissues may be useful for identifying potential therapeutic agents for treating a disease. In contrast to targeting a single protein or pathway, targeting whole genome states that are specific to certain diseases will allow for precision medicine, better specificity in treating diseased cells, and minimization of drug resistance. Also provided are methods of using compounds identified by the present screening methods for such targeting of whole genome states that are specific to certain diseases. In certain embodiments, provided are methods of targeting whole chromatin states specific to a disease by organizing the chromatin state into a phase-separated condensate. According to some embodiments, provided are methods that reduce drug resistance and/or side effects by targeting whole chromatin states, e.g., whole chromatin states specific to a disease.

Diseases associated with epigenetic changes and altered chromatin states include, without limitation, Fragile X syndrome, Angelman's syndrome, Prader-Willi syndrome, various cancers, neurodegenerative diseases, including Huntington's disease, Alzheimer's disease, Parkinson's disease, and schizophrenia; autoimmune diseases, including systemic lupus erythematosus, rheumatoid arthritis, systemic sclerosis, Sjogren's syndrome, autoimmune thyroid diseases, type 1 diabetes, and asthma; and cardiovascular diseases, including atherosclerosis and cardiomyopathies.

The nucleosome array condensate may be contacted with agents by any convenient means. Generally, an agent is added to the nucleosome array condensate such that the agent is brought in contact with the phase-separated chromatin at an effective concentration to produce a desired effect. The effective concentration of an agent will vary and will depend on the agent. In some embodiments, the effective concentration of agents ranges from 1 ng/mL to 10 mg/mL or more, including but not limited to, e.g., 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, 25 ng/mL, 26 ng/mL, 27 ng/mL, 28 ng/mL, 29 ng/mL, 30 ng/mL, 31 ng/mL, 32 ng/mL, 33 ng/mL, 34 ng/mL, 35 ng/mL, 36 ng/mL, 37 ng/mL, 38 ng/mL, 39 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 1-5 ng/mL, 1-10 ng/mL, 1-20 ng/mL, 1-30 ng/mL, 1-40 ng/mL, 1-50 ng/mL, 5-10 ng/mL, 5-20 ng/mL, 10-20 ng/mL, 10-30 ng/mL, 10-40 ng/mL, 10-50 ng/mL, 20-30 ng/mL, 20-40 ng/mL, 20-50 ng/mL, 30-40 ng/mL, 30-50 ng/mL, 40-50 ng/mL, 1-100 ng/mL, 50-100 ng/mL, 60-100 ng/mL, 70-100 ng/mL, 80-100 ng/mL, 90-100 ng/mL, 10-100 ng/mL, 50-200 ng/mL, 100-200 ng/mL, 50-300 ng/mL, 100-300 ng/mL, 200-300 ng/mL, 50-400 ng/mL, 100-400 ng/mL, 200-400 ng/mL, 300-400 ng/mL, 50-500 ng/mL, 100-500 ng/mL, 200-500 ng/mL, 300-500 ng/mL, 400 to 500 ng/mL, 0.001-1 μg/mL, 0.001-2 μg/mL, 0.001-3 μg/mL, 0.001-4 μg/mL, 0.001-5 μg/mL, 0.001-6 μg/mL, 0.001-7 μg/mL, 0.001-8 μg/mL, 0.001-9 μg/mL, 0.001-10 μg/mL, 0.01-1 μg/mL, 0.01-2 μg/mL, 0.01-3 μg/mL, 0.01-4 μg/mL, 0.01-5 μg/mL, 0.01-6 μg/mL, 0.01-7 μg/mL, 0.01-8 μg/mL, 0.01-9 μg/mL, 0.01-10 μg/mL, 0.1-1 μg/mL, 0.1-2 μg/mL, 0.1-3 μg/mL, 0.1-4 μg/mL, 0.1-5 μg/mL, 0.1-6 μg/mL, 0.1-7 μg/mL, 0.1-8 μg/mL, 0.1-9 μg/mL, 0.1-10 μg/mL, 0.5-1 μg/mL, 0.5-2 μg/mL, 0.5-3 μg/mL, 0.5-4 μg/mL, 0.5-5 μg/mL, 0.5-6 μg/mL, 0.5-7 μg/mL, 0.5-8 μg/mL, 0.5-9 μg/mL, 0.5-10 μg/mL, 0.1 mg/mL-10 mg/mL, 0.1 mg/mL-1 mg/mL, 1 mg/mL-9 mg/mL, 2 mg/mL-8 mg/mL, 3 mg/mL-7 mg/mL, and the like.

The effect of an agent is determined by adding the agent to the nucleosome array condensate and monitoring one or more parameters usually with comparison to a control nucleosome array condensate lacking the agent. The parameters may include, without limitation, dissolution of condensates, number of condensates, size of condensates, number or frequency of histone modifications (e.g., lysine and arginine methylation, lysine acetylation, serine and threonine phosphorylation, sumoylation, or ubiquitination) or DNA modifications (e.g., methylation), or combinations thereof. While most parameters will provide a quantitative readout, in some instances a semi-quantitative or qualitative result will be acceptable. In certain embodiments, one or more heterochromatin proteins are labeled, wherein assessing the nucleosome array condensate for a modulation of the chromatin state by the agent comprises detecting the labeled heterochromatin proteins. In other embodiments, the nucleosome array condensate comprises labeled DNA, wherein assessing the nucleosome array condensate for a modulation of the chromatin state by the agent comprises detecting the labeled DNA. Readouts may include a single determined value, or may include a mean, median value or variance, etc. Characteristically a range of parameter readout values will be obtained for each parameter from a multiplicity of the same assays. Some variability is expected and a range of values for each set of test parameters may be obtained and analyzed using standard statistical methods.

The nucleosome array condensates can be monitored optically by any suitable method. For example, images of nucleosome array condensates can be obtained using a microscope, such as a confocal microscope, a light microscope, a fluorescence microscope, an inverted microscope, a digital microscope, or other high magnification imaging system. Any optical method may be used, such as bright field, dark field, phase contrast, Hoffman modulation contrast, fluorescence, or differential interference contrast. In certain embodiments, a digital camera may be used to capture images of the nucleosome array condensates. The camera may be coupled to a computer for receiving and processing digital data from the digital camera. The image of the nucleosome array condensates may be a still photo or a video in any format (e.g., bitmap, Graphics Interchange Format, JPEG file interchange format, TIFF, or mpeg). Alternatively, the image of the nucleosome array condensates may be captured by an analog camera and converted into an electronic form.

For screening of multiple nucleosome array condensates in parallel, the condensates may be placed in separate containers (e.g., tubes of a multi-tube rack or wells of a multi-well plate) and imaged after contacting the condensates with candidate agents. Imaging may be performed on nucleosome array condensates in suspension. Alternatively, nucleosome array condensates may be allowed to settle to the bottom of a container and imaged through the bottom. In certain embodiments, the bottom of the container is transparent to facilitate microscopic visualization or imaging of the nucleosome array condensates from the bottom. Imaging parameters should be held constant across samples to allow comparison of multiple nucleosome array condensates.

In some embodiments, methods are provided for screening candidate agents in a high-throughput format. By “high-throughput” is meant the screening of large numbers of candidate agents simultaneously for an activity of interest. By large numbers, it is meant screening 20 more or candidates at a time, e.g., 40 or more candidates, e.g., 100 or more candidates, 200 or more candidates, 500 or more candidates, or 1000 candidates or more.

In some embodiments, the high-throughput screen will be formatted based upon the numbers of wells in multi-well plates that are used, e.g. a 24-well format, in which 24 candidate agents (or less, plus controls) are assayed; a 48-well format, in which 48 candidate agents (or less, plus controls) are assayed; a 96-well format, in which 96 candidate agents (or less, plus controls) are assayed; a 384-well format, in which 384 candidate agents (or less, plus controls) are assayed; a 1536-well format, in which 1536 candidate agents (or less, plus controls) are assayed; or a 3456-well format, in which 3456 candidate agents (or less, plus controls) are assayed.

Candidate agents of interest for screening include known and unknown compounds that encompass numerous chemical classes, primarily organic molecules, which may include organometallic molecules, inorganic molecules, genetic sequences, etc. Candidate agents may include organic molecules comprising functional groups necessary for structural interactions, particularly electrostatic or hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents may include hydroxamates, carbazoles, anthracyclines, acridines, fatty acids, intercalators, DNA major and minor groove binders, alkylating agents, and analogues of the metabolite S-adenosylmethionine (SAM). Candidate agents are also found among biomolecules, including peptides, proteins, antibodies, polynucleotides, saccharides, fatty acids, steroids, purines, pyrimidines, derivatives, structural analogs or combinations thereof. Included are pharmacologically active drugs, genetically active molecules, etc. Exemplary pharmaceutical agents include those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Compounds of interest include methyltransferase inhibitors, acetyltransferase inhibitors, ubiquitin ligase inhibitors, demethylase inhibitors, histone deacetylase (HDAC) inhibitors, histone acetyltransferase inhibitors, bromodomain inhibitors, histone demethylase inhibitors, histone methyltransferase inhibitors, histone acetyl reader inhibitors, histone methyl reader inhibitors, histone ubiquitin ligase inhibitors, DNA methyltransferase (DNMT) inhibitors, and topoisomerase inhibitors; chromatin modifying enzymes such as enzymes that modify genomic DNA methylation patterns, including DNA methyltransferases; histone-modifying enzymes, including histone deacetylases, histone acetyltransferases, histone methyltransferases, and histone lysine demethylases; enzymes that regulate chromatin topology, including DNA topoisomerases; and other chromatin modifiers.

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for their effects on chromatin by adding the agent to at least one nucleosome array condensate, usually in conjunction with a control condensate that is not contacted with the agent. Changes in parameters in response to the agent are measured, and the result is evaluated by comparison to reference condensates, which may include condensates in the presence or absence of the agent, or treated with other agents, etc.

The agents are conveniently added in solution or as a readily soluble form to chromatin droplets. The agents can be injected into a chromatin droplet, and their effects compared to injection of controls. Alternatively, the agents may be added in a flow-through system, as a stream, intermittent or continuous, or alternatively, adding a bolus of the compound, singly or incrementally, to an otherwise static solution.

Preferred agent formulations do not include additional components, such as preservatives, which may have a significant effect on the results. Thus, preferred formulations consist essentially of a candidate compound and may include an acceptable carrier, e.g., water, ethanol, DMSO, etc. If a compound is a liquid, it may not require a solvent, and the formulation may consist essentially of the compound itself.

A plurality of assays may be run in parallel with different agent concentrations to obtain a differential response to the various concentrations. As known in the art, determining the effective concentration of an agent typically involves testing a range of concentrations resulting from 1:10, or other log scale, dilutions. The amount of an agent needed to be effective may be further refined with a further series of dilutions, if necessary. A control may include the agent at zero concentration, at a concentration below the level of detection of the agent, or at a concentration that does not give a detectable change in the parameters used to monitor the nucleosome array condensates.

A candidate agent may also be screened for efficacy in treating or preventing a disease. In such embodiments, the nucleosome array condensate models a disease-associated chromatin state. For example, screening may involve monitoring chromatin parameters to identify a compound that modulates the disease-associated chromatin state and/or restores a normal chromatin state. A candidate agent may also be screened for toxicity to cells or tissue. In these applications, cells are exposed to a candidate agent and their growth and viability are assessed.

### Systems and Computer Implemented Methods

The present disclosure also provides systems which find use in practicing the subject methods. In some embodiments, the system may include a processor programmed to process fluorescence images of fluorescently labeled nucleosome array condensates; and a display component for displaying information about the nucleosome array condensates. Such information may include the size distributions of the nucleosome array condensates, shape distributions of the nucleosome array condensates, quantification of the nucleosome array condensates, histone modifications or DNA modifications, or any combination thereof.

In some embodiments, a computer implemented method is used for processing fluorescence images of fluorescently labeled nucleosome array condensates. The processor may be programmed to perform steps of the computer implemented method comprising: a) receiving the fluorescence images of the fluorescently labeled nucleosome array condensates; b) performing segmentation-free image quantification on the fluorescence images; c) computing size distributions of the nucleosome array condensates from the fluorescence images; d) reducing complexities of the size distributions into distinct features that discriminate between the fluorescence images to simplify quantification of the nucleosome array condensates; e) acquiring shape distributions for the nucleosome array condensates from the fluorescence images; and f) reducing complexities of the shape distributions into distinct features that discriminate between the fluorescence images to simplify quantification of the nucleosome array condensates. In some embodiments, the computer implemented method further comprises quantifying z-factors using the outputs from steps d) and f) to assess quality control and reproducibility. In some embodiments, the computer implemented method further comprises comparing data from multiple screens to identify test agents that specifically modulate the chromatin state of interest.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, the system for performing the computer implemented method, as described, may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions. For example, the storage component includes instructions for processing fluorescence images of fluorescently labeled nucleosome array condensates, as described herein (e.g., see Examples). The computer processor is coupled to the storage component and configured to execute the instructions stored in the storage component in order to receive fluorescence images of the fluorescently labeled nucleosome array condensates and analyze the fluorescence image data according to one or more algorithms, as described herein. The display component displays information about the nucleosome array condensates that is useful for distinguishing nucleosome array condensates that have different chromatin states. For example, the display component may display information regarding the size distributions of the nucleosome array condensates, shape distributions of the nucleosome array condensates, and quantification of the nucleosome array condensates. The display may also display information about the number or frequency of histone modifications (e.g., lysine and arginine methylation, lysine acetylation, serine and threonine phosphorylation, sumoylation, or ubiquitination) and/or DNA modifications (e.g., methylation).

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel.

### Kits

Also provided are kits comprising nucleosome array condensates or reagents for producing nucleosome array condensates, as described herein. In certain embodiments, the kit includes a multi-well plate suitable for high-throughput screening of candidate agents. Such a multi-well plate may comprise at least 2, at least 4, at least 6, at least 12, at least 24, at least 48, at least 96, at least 384, or at least 1536 wells. The kit may also include reagents or equipment for imaging nucleosome array condensates.

In some embodiments, the kit comprises software for carrying out the computer implemented methods for processing fluorescence images of fluorescently labeled nucleosome array condensates, as described herein. In some embodiments, the kit comprises a system for distinguishing nucleosome array condensates that have different chromatin states, as described herein. Such a system may comprise: a processor programmed to process fluorescence images of fluorescently labeled nucleosome array condensates according to the computer implemented method described herein; and a display component for displaying information about the nucleosome array condensates (e.g., regarding the size distributions of the nucleosome array condensates, shape distributions of the nucleosome array condensates, quantification of the nucleosome array condensates, and modifications of histones and DNA). In certain embodiments, the system further comprises reagents for forming a phase-separated droplet comprising a fluorescently labeled nucleosome array condensate. In certain embodiments, the system further comprises a test agent.

In addition to the above components, the subject kits may further include (in certain embodiments) instructions for practicing the subject methods. In some embodiments, instructions for using the nucleosome array condensate for screening for candidate agents that modulate chromatin or a disease-associated chromatin state are provided in the kits. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, SD drive, and the like, on which the information has been recorded. Yet another form of these instructions that may be present is a website address which may be used via the internet to access the information at a removed site.

Notwithstanding the appended claims, the present disclosure is also defined by the following embodiments:

1. A method of assessing a test agent for the ability to modulate a chromatin state of interest, the method comprising:
contacting a phase-separated droplet comprising a nucleosome array condensate with the test agent; and
assessing the nucleosome array condensate for a modulation of the chromatin state of interest by the test compound.

2. The method according to embodiment 1, wherein the test agent is a small molecule, a biomolecule, or a polypeptide.

3. The method according to embodiment 1 or 2, wherein the chromatin state of interest mimics a naturally occurring chromatin state.

4. The method according to embodiment 3, wherein the naturally occurring chromatin state is a chromatin state of a normal cell.

5. The method according to embodiment 3, wherein the naturally occurring chromatin state is a chromatin state associated with a disease.

6. The method according to embodiment 5, wherein the disease is cancer, a neurodegenerative disease, or an autoimmune disease.

7. The method according to embodiment 6, wherein the chromatin state is specific to a type of cancer.

8. The method according to embodiment 7, wherein the cancer is diffuse intrinsic pontine glioma (DIPG).

9. The method according to any one of embodiments 1 to 8, wherein the nucleosome array condensate comprises histone modifications.

10. The method according to embodiment 9, wherein the histone modifications comprise one or more histone modifications selected from the group consisting of: H3K9me3, H4K20me3, H3K27me3, H3K4me3, H3K36me3, H3K79me3, H3K56ac, H3K9Ac, H4K16Ac, H4K14Ac, H4K18Ac, and any combination thereof.

11. The method according to embodiment 9 or embodiment 10, wherein the one or more histone modifications have a nucleosome occupancy selected to mimic a naturally occurring chromatin state.

12. The method according to embodiment 11, wherein the nucleosome occupancy is 60% or greater.

13. The method according to any one of embodiments 1 to 12, wherein the nucleosome array condensate comprises one or more histone variants.

14. The method according to embodiment 13, wherein the one or more histone variants comprise H2Az, macro H2A, H3.3, CENP-A, H2ABBD, or any combination thereof.

15. The method according to any one of embodiments 9 to 14, wherein the nucleosome array condensate comprises one or more heterochromatin or euchromatin proteins bound to the one or more histone modifications.

16. The method according to embodiment 15, wherein the one or more heterochromatin or euchromatin proteins bind cooperatively to the nucleosome array condensate.

17. The method according to embodiment 15 or 16, wherein the one or more heterochromatin or euchromatin proteins comprise chromobox protein homolog 5 (CBX5), chromobox protein homolog 3 (CBX3), chromobox protein homolog 1 (CBX1), polycomb repressive complex 1 (PRC1), polycomb repressive complex 2 (PRC2), or any combination thereof.

18. The method according to any one of embodiments 15 to 17, wherein the one or more heterochromatin or euchromatin proteins exhibit a specificity for histones of the nucleosome array of 2-fold or greater as compared to the specificity for histones of a corresponding nucleosome array in which the one or more histone modifications are absent.

19. The method according to any one of embodiments 15 to 18, wherein the one or more heterochromatin or euchromatin proteins exhibit a dissociation constant (K_{D}) for the nucleosome array of less than or equal to 5 μM.

20. The method according to any one of embodiments 15 to 19, wherein the one or more heterochromatin or euchromatin proteins are labeled, and wherein assessing the nucleosome array condensate for a modulation of the chromatin state of interest by the test compound comprises detecting the labeled heterochromatin or euchromatin proteins.

21. The method according to any one of embodiments 1 to 20, wherein the nucleosome array condensate comprises one or more proteins that associate with chromatin or DNA.

22. The method according to embodiment 21, wherein the one or more proteins that associate with chromatin or DNA comprise a polycomb-group protein, a bromodomain-containing protein, a chromobox protein homolog, a mediator complex subunit, or an oncogenic fusion protein, or any combination thereof.

23. The method according to embodiment 22, wherein the polycomb-group protein is PRC1 or PRC2, the bromodomain-containing protein is BRD1, BRD2, BRD3, BRD4, BRD5, BRD6, BRD7, BRD8, or BRD9, the chromobox protein homolog is CBX1, CBX2, CBX3, CBX4, CBX5, CBX6, CBX7, or CBX8, and the oncogenic fusion protein is AML-ETO.

24. The method according to any one of embodiments 1 to 23, wherein the nucleosome array condensate comprises labeled DNA, and wherein assessing the nucleosome array condensate for a modulation of the chromatin state of interest by the test compound comprises detecting the labeled DNA.

25. The method according to any one of embodiments 1 to 24, wherein the DNA used to assemble the nucleosome array is custom-designed to position nucleosomes at specific locations.

26. The method according to any one of embodiments 1 to 24, wherein the DNA used to assemble the nucleosome array comprises naturally occurring DNA sequences.

27. The method according to any one of embodiments 1 to 26, wherein the method is used to screen for and identify test agents that affect cooperativity or binding of proteins known to associate with chromatin or DNA or phase separation properties of chromatin.

28. The method according to any one of embodiments 1 to 27, further comprising counter-screening against other types of chromatin states to identify test agents that specifically modulate the chromatin state of interest.

29. The method according to any one of embodiments 1 to 28, wherein the method is a cell-free method.

30. The method according to any one of embodiments 1 to 29, wherein assessing the nucleosome array condensate for a modulation of the chromatin state of interest comprises assessing the nucleosome array condensate for a modulation of dissolution, shape change, size change, or any combination thereof.

31. A method to distinguish nucleosome array condensates that have different chromatin states, the method comprising:
a) performing fluorescence imaging of fluorescently labeled nucleosome array condensates to obtain fluorescence images;
b) performing segmentation-free image quantification on the fluorescence images;
c) computing size distributions of the nucleosome array condensates from the fluorescence images;
d) reducing complexities of the size distributions into distinct features that discriminate between the fluorescence images to simplify quantification of the nucleosome array condensates;
e) acquiring shape distributions for the nucleosome array condensates from the fluorescence images; and
f) reducing complexities of the shape distributions into distinct features that discriminate between the fluorescence images to simplify quantification of the nucleosome array condensates.

32. The method according to embodiment 31, further comprising quantifying z-factors using the outputs from steps d) and f) to assess quality control and reproducibility.

33. The method according to embodiment 31 or 32, further comprising comparing data from multiple screens to identify test agents that specifically modulate the chromatin state of interest.

34. A computer implemented method for processing fluorescence images of fluorescently labeled nucleosome array condensates, the computer performing steps comprising:
a) receiving the fluorescence images of the fluorescently labeled nucleosome array condensates;
b) performing segmentation-free image quantification on the fluorescence images;
c) computing size distributions of the nucleosome array condensates from the fluorescence images;
d) reducing complexities of the size distributions into distinct features that discriminate between the fluorescence images to simplify quantification of the nucleosome array condensates;
e) acquiring shape distributions for the nucleosome array condensates from the fluorescence images; and
f) reducing complexities of the shape distributions into distinct features that discriminate between the fluorescence images to simplify quantification of the nucleosome array condensates.

35. The method according to embodiment 34, further comprising quantifying z-factors using the outputs from steps d) and f) to assess quality control and reproducibility.

36. The method according to embodiment 34 or 35, further comprising comparing data from multiple screens to identify test agents that specifically modulate the chromatin state of interest.

37. A non-transitory computer-readable medium comprising program instructions that, when executed by a processor in a computer, causes the processor to perform the method of any one of embodiments 34 to 36.

38. A system for distinguishing nucleosome array condensates that have different chromatin states, the system comprising:
a processor programmed to process fluorescence images of fluorescently labeled nucleosome array condensates according to the computer implemented method of any one of embodiments 34 to 36; and
a display component for displaying information about the nucleosome array condensates regarding size distributions of the nucleosome array condensates, shape distributions of the nucleosome array condensates, quantification of the nucleosome array condensates, or histone modifications or DNA modifications, or any combination thereof.

39. The system according to embodiment 38, further comprising reagents for forming a phase-separated droplet comprising a fluorescently labeled nucleosome array condensate.

40. The system according to embodiment 38 or 39, further comprising a test agent.

41. A pharmaceutical composition comprising a test agent identified as modulating a chromatin state of interest by the method of any one of embodiments 1 to 28.

42. A method comprising administering to an individual in need thereof an effective amount of a test agent identified as modulating a chromatin state of interest by the method of any one of embodiments 1 to 28.

43. A method of producing a nucleosome array condensate that mimics a naturally occurring chromatin state, the method comprising: combining one or more heterochromatin or euchromatin proteins and a nucleosome array under conditions in which the nucleosome array forms a nucleosome array condensate that mimics a naturally occurring chromatin state, wherein the nucleosome array comprises histone modifications, histone variants, or both, selected to mimic the naturally occurring chromatin state.

44. The method according to embodiment 43, wherein the one or more heterochromatin or euchromatin proteins comprise CBX5, CBX3, CBX1, PRC1, PRC2, or any combination thereof.

45. The method according to embodiment 43 or embodiment 44, wherein the one or more heterochromatin or euchromatin proteins exhibit a specificity for histones of the nucleosome array of 2-fold or greater as compared to the specificity for histones of a corresponding nucleosome array in which the one or more histone modifications and/or histone variants are absent.

46. The method according to any one of embodiments 43 to 45, wherein the one or more heterochromatin or euchromatin proteins exhibit a dissociation constant (K_{D}) for the nucleosome array of less than or equal to 5 μM.

47. The method according to any one of embodiments 43 to 46, wherein the one or more heterochromatin or euchromatin proteins are labeled.

48. The method according to any one of embodiments 43 to 47, wherein the nucleosome array comprises histone modifications comprising H3K9me3, H4K20me3, H3K27me3, H3K4me3, H3K36me3, H3K79me3, H3K56ac, H3K9Ac, H4K16Ac, H4K14Ac, H4K18Ac, or any combination thereof, selected to mimic the naturally occurring chromatin state.

49. The method according to any one of embodiments 43 to 48, wherein the nucleosome array comprises histone variants comprising H2Az, macro H2A, H3.3, CENP-A, H2ABBD, or any combination thereof, selected to mimic the naturally occurring chromatin state.

50. The method according to any one of embodiments 43 to 49, further comprising adding one or more proteins that associate with chromatin or DNA to the nucleosome array condensate, wherein the one or more proteins that associate with chromatin or DNA comprise a polycomb-group protein, a bromodomain-containing protein, a chromobox protein homolog, a mediator complex subunit, or an oncogenic fusion protein, or any combination thereof, selected to mimic the naturally occurring chromatin state.

51. The method according to embodiment 50, wherein the polycomb-group protein is PRC1 or PRC2, the bromodomain-containing protein is BRD1, BRD2, BRD3, BRD4, BRD5, BRD6, BRD7, BRD8, or BRD9, the chromobox protein homolog is CBX1, CBX2, CBX3, CBX4, CBX5, CBX6, CBX7, or CBX8, and the oncogenic fusion protein is AML-ETO.

52. The method according to any one of embodiments 43 to 51, wherein the nucleosome array comprises labeled DNA.

53. The method according to any one of embodiments 43 to 52, wherein the method is a cell-free method.

### EXAMPLES

The following examples are put forth so as to provide those of ordinary skill in the art with a complete disclosure and description of how to make and use the disclosed subject matter, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1: Methods for Generating Biologically Specific Chromatin Condensates

### Materials and Methods

### F40 Sortase Purification

Recombinant F40 sortase was expressed from BL21(DE3) cells and purified as previously described (Wu et al. eLIFE 2018).

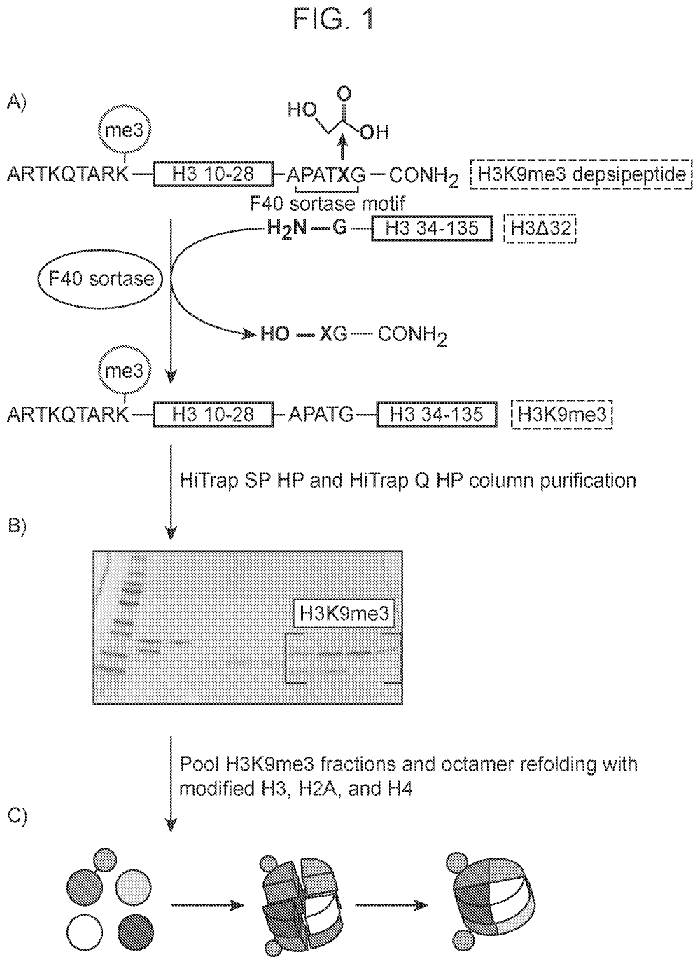

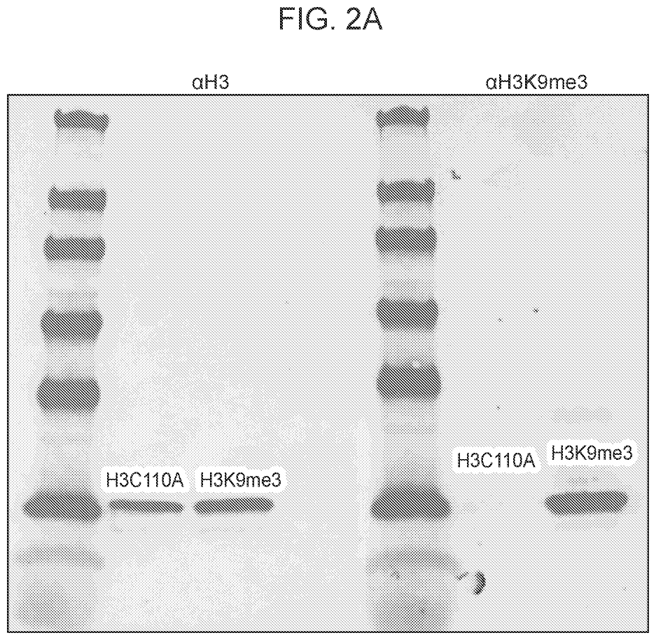

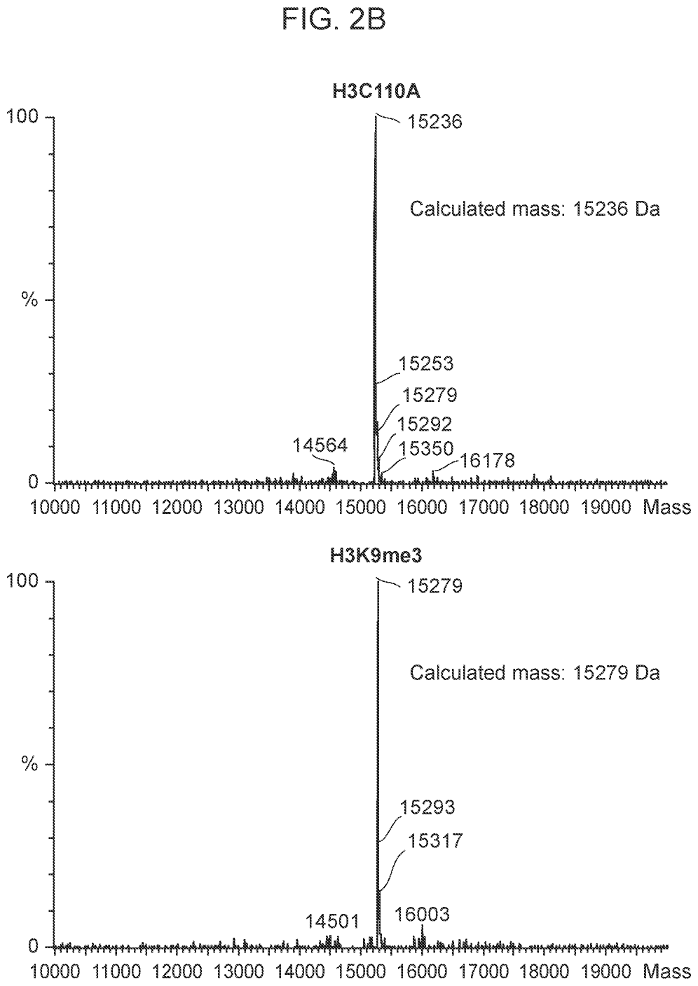

### Histone Purification and Semi-Synthesis (FIG. 1)

All histones were expressed from BL21(DE3) pLysS cells and purified as previously published (Luger et al. Methods Enzymol. 1999 and web protocol from Tsukiyama Lab). To make native modifications on H3, H3Δ32 was purified and the H3 semi-synthesis was performed as previously described (Wu et al. eLIFE 2018) with the following modifications: 80 μM H3Δ32 histone was mixed with 1.2 mM modified H3 depsipeptide (aa1-31) (GenScript), and 300 μM F40 sortase in 50 mM HEPES (pH 7.5), 150 mM NaCL, 5 mM CaCl₂, and 1 mM DTT at 37° C. for 18 hours. The pellet was resuspended in 20 mM Tris (pH7.8), 1 mM EDTA, 7 M urea, 10 mM NaCl, and 5 mM BME and run over a HiTrap Q HP in tandem with a HiTrap SP HP column, and eluted off the HiTrap SP HP. The ligated H3 with the modification was confirmed using LC/MS and by Western blot with antibodies recognizing the modifications (Abcam).

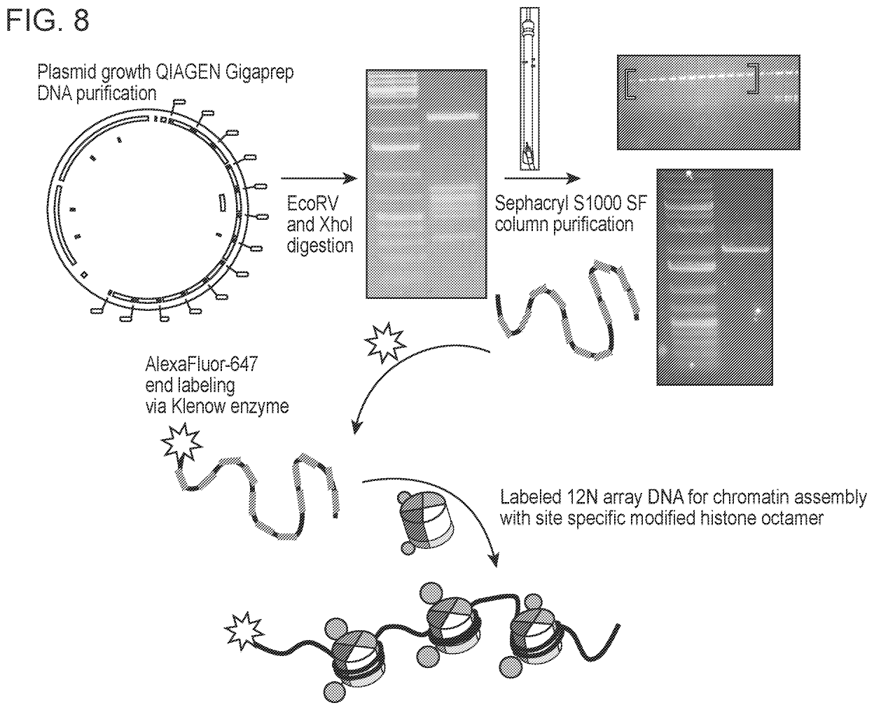

### S1000 Column Preparation (FIG. 8, Top Part)

The Sephacryl S1000 super fine resin slurry (Cytiva) was mixed and added into an empty XK 26/600 column (Cytiva) and residual liquid was drained at room temperature until the liquid covered the resin. This was repeated until the resin reached the top of the column. The column was hooked up to an FPLC and 1 CV of water was run on the column at a flow rate of 3 ml/min to pack the resin. After the first round of packing, more resin was added until and packed at 3 ml/min until the resin filled the top of the column. A total of ˜750 ml of S1000 slurry was needed to fully pack the column.

### 12×601 Array DNA Purification and Labeling (FIG. 8, Top Part)

Plasmid containing the 12×601 array DNA (Gibson et al. Cell 2019) was propagated in Stbl2 cells (NEB) and was purified using the GigaPrep Kit (Qiagen). To isolate the 12×601 array DNA, the plasmid was digested with restriction enzymes EcoRV and XhoI (NEB) at 37° C. for 18 hours, and purified by gel filtration using a custom packed S1000 column. The DNA was subsequently ethanol precipitated and eluted in 1×TE.

Array DNA was cut with XhoI to create a 5′ overhang for fluorescent nucleotide labeling with Klenow Fragment 3′-5′ exo-(NEB). Purified array DNA (1 mg/mL) was incubated with the Klenow fragment (0.032 U/μg), 35 μM dATP, dTTP, dGTP, Alexa Fluor 647-aha-dCTP (ThermoScientific), and 1×NEBuffer 2 overnight at room temperature, covered in foil. Labeled DNA was subsequently purified by phenol-chloroform extraction and ethanol precipitation, and resuspended in 1×TE at 4 mg/mL.

### Chromatin Assembly (FIG. 8, Bottom Part)

Histones were refolded in high salt buffer to form octamers and purified by size exclusion chromatography as previously published (Luger et al. Methods Enzymol. 1999). Nucleosome arrays were assembled using salt gradient dialysis as previously described (Sanulli et al. Nature 2019).

### HP1α Purification

Human pBH4-HP1α was transformed into Rosetta BL21 (DE3) competent cells in E. coli. Cultures were grown at 30° C. in 2×LB with 50 μg/ml carbenicillin and 25 μg/ml chloramphenicol until OD˜0.4 was reached. Cells were then transferred to 18° C. and grown to OD˜0.8 before inducing expression with 0.4 mM isopropyl-OD-thiogalactopyranoside for 16 hours. Cells were harvested by centrifugation at 4000 g for 30 mins and resuspended in lysis buffer (1×PBS, 300 mM KCl, 10% glycerol, 7.5 mM imidazole, and protease inhibitors—100 mM phenylmethanesulfonyl fluoride, 2 μg/ml Aprotinin, 3 μg/ml Leupeptin, and 1 μg/ml Pepstatin A). The cells were then lysed using the C3 Emulsiflex, and clarified lysate was obtained by centrifugation at 25000 g for 30 mins. Clarified lysate was incubated with Talon cobalt resin for 1 hour at 4° C. with rocking. The resin-lysate solution was added to a gravity column and washed with ˜50 mls lysis buffer without protease inhibitors and eluted with 10 mls elution buffer (20 mM HEPES pH 7.2, 100 mM KCl, 400 mM imidazole). Protein was dialyzed overnight at 4° C. with 0.5 mg TEV protease/L culture in dialysis buffer (20 mM HEPES pH 7.2, 150 mM KCl, 3 mM DTT) to cleave off the 6×-His tag and to remove imidazole. The cleaved protein was then injected onto a Mono-Q 10/100 GL anion exchange column (GE) and eluted with a 150-800 mM KCl gradient over 15 column volumes. Clean fractions were pooled and concentrated in an Amicon Ulta 10K spin concentrator before final injection onto a Superdex S75 Increase 10/300 GL column running with size-exclusion/storage buffer (20 mM HEPES pH 7.2, 300 mM KCl, 10% glycerol, 3 mM DTT). Desired fractions were pooled, concentrated, and flash frozen in liquid nitrogen for long term storage at −80° C.

### HP1α Labeling

pBH4-HP1α was modified with a “GSKCK” tag at the C-terminal end, and cysteine 133 was mutated to a serine to encourage specific labeling. HP1α-KCK was purified following the HP1α purification method. Before labeling, the protein was dialyzed in labeling buffer overnight (20 mM HEPES pH 7.2, 350 mM KCl, 10% glycerol, 0.5 mM TCEP) at 4° C. Protein concentration was then adjusted to 200 μM and mixed with Cy3 maleimide at a 1:1 molar ratio. The reaction was immediately quenched (˜5 secs) with 10× molar excess β-mercaptoethanol. Free dye was separated from labeled protein using an ILLUSTRA G50 column following the manufacturer's protocol. Labeled protein was then flash frozen in liquid nitrogen and stored at −80 C. Labeling efficiency was determined by comparing Cy3 dye absorption to HP1α concentration. 150,000M⁻¹cm⁻¹ at 552 nm was used for Cy3, and 29,495 M⁻¹cm⁻¹ at 280 nm was used for HP1α.

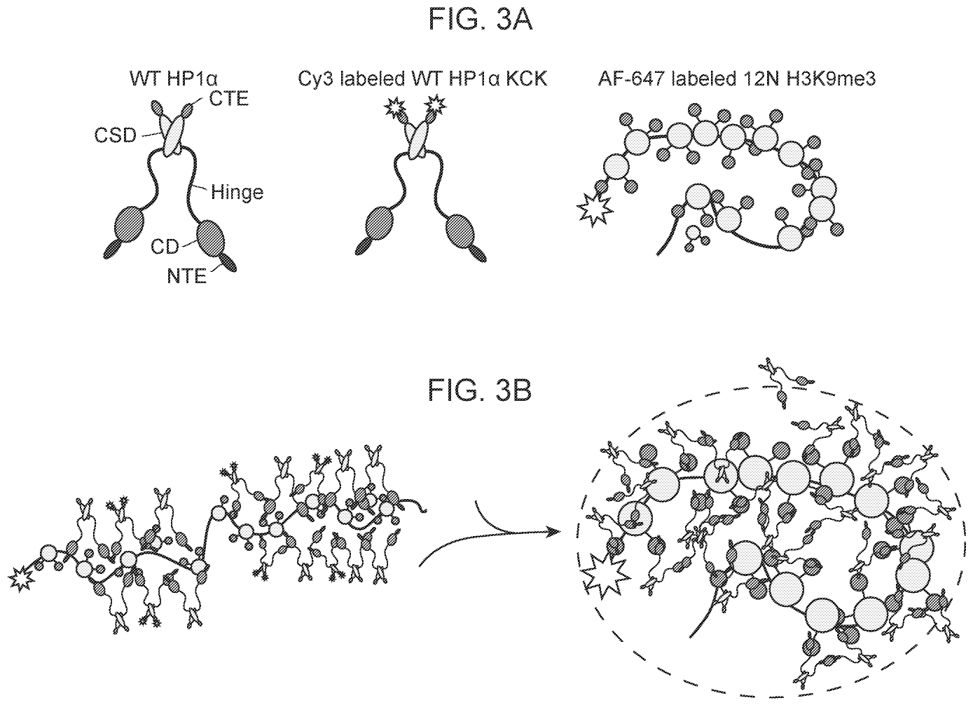

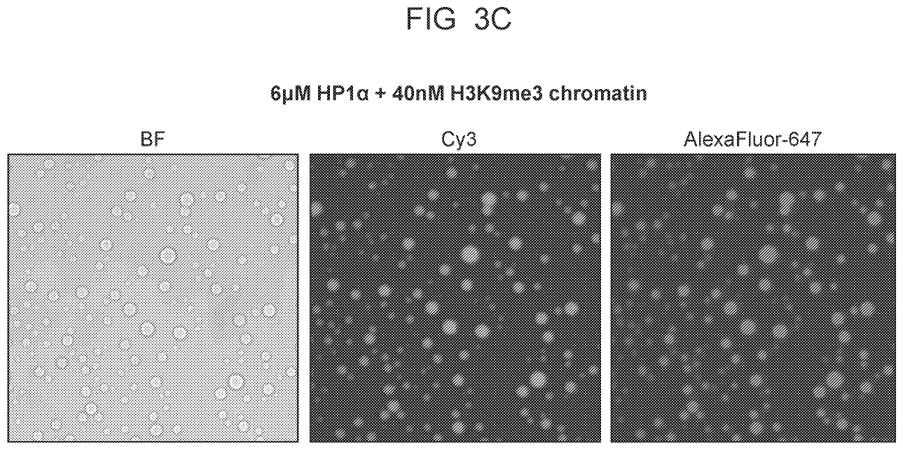

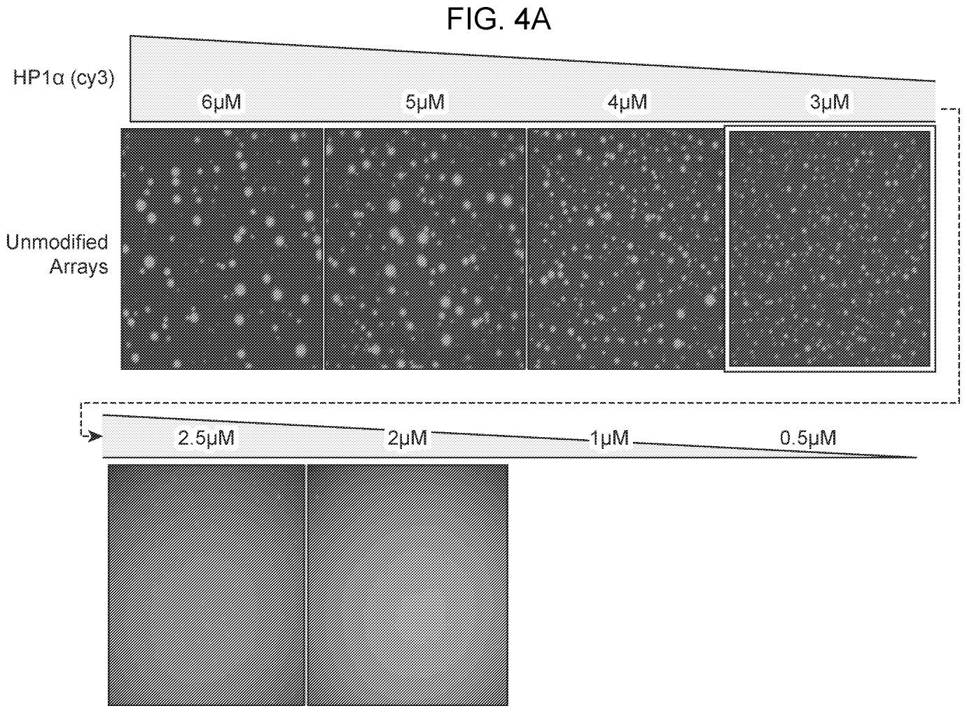

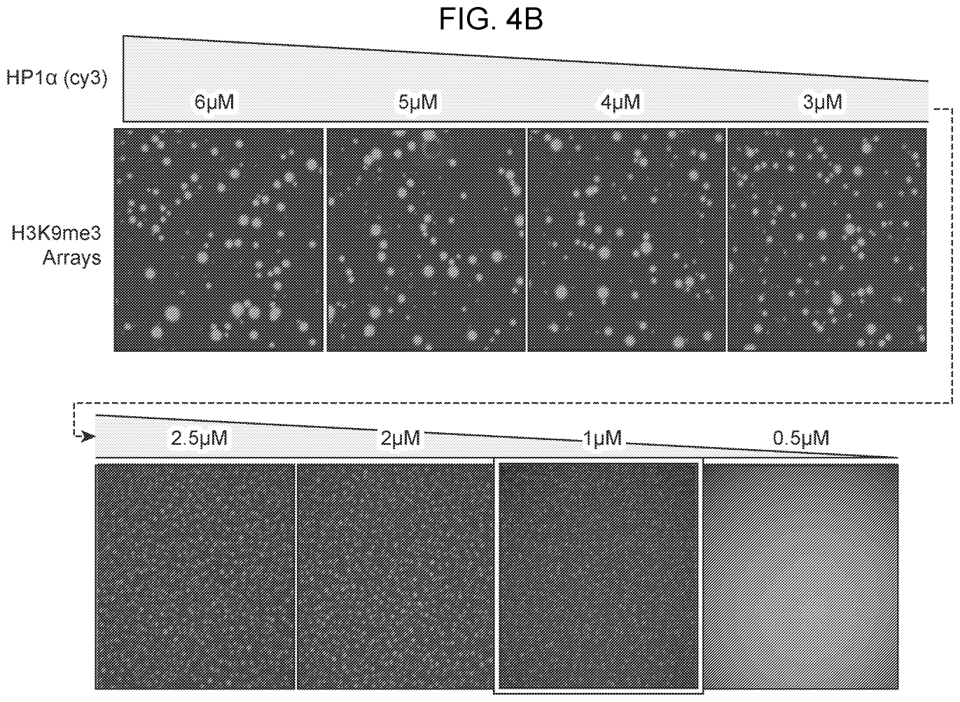

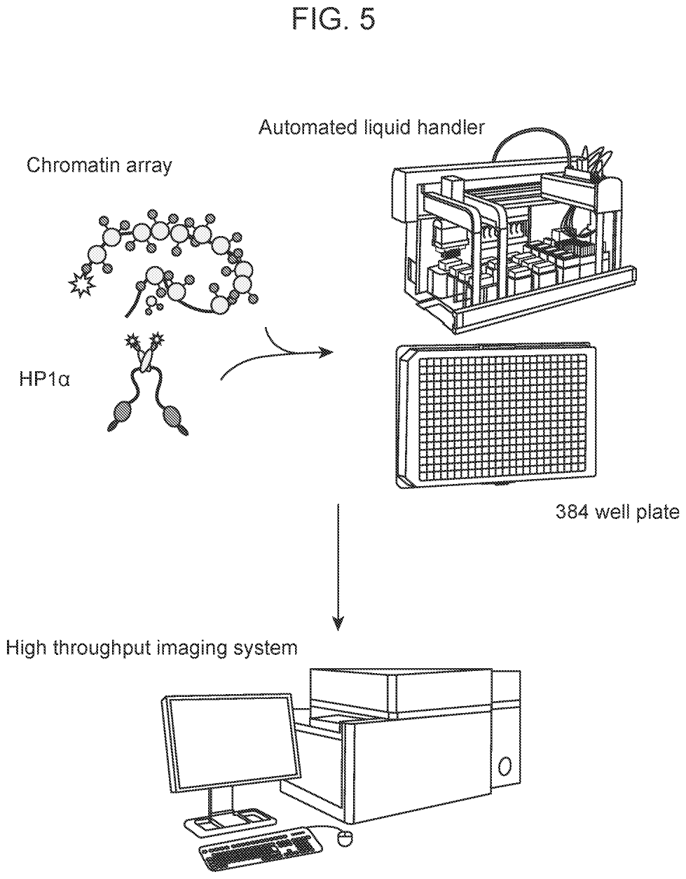

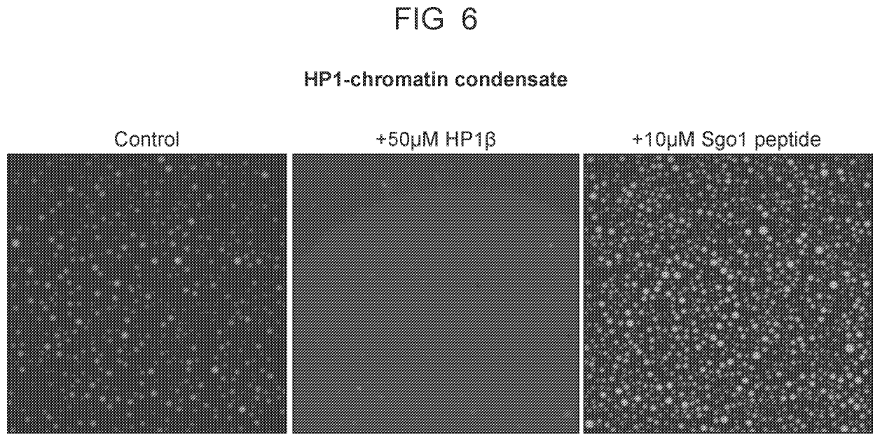

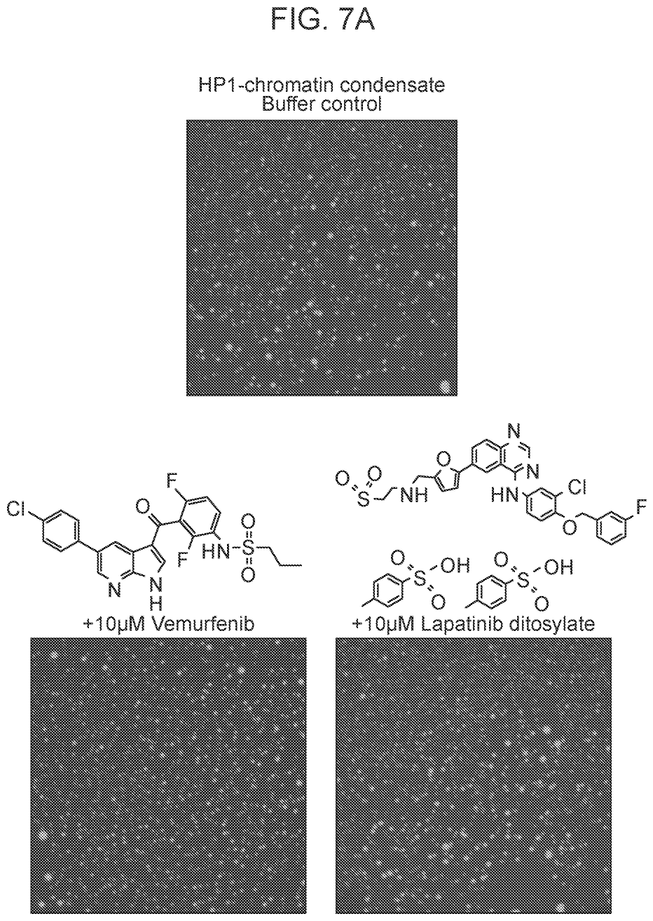

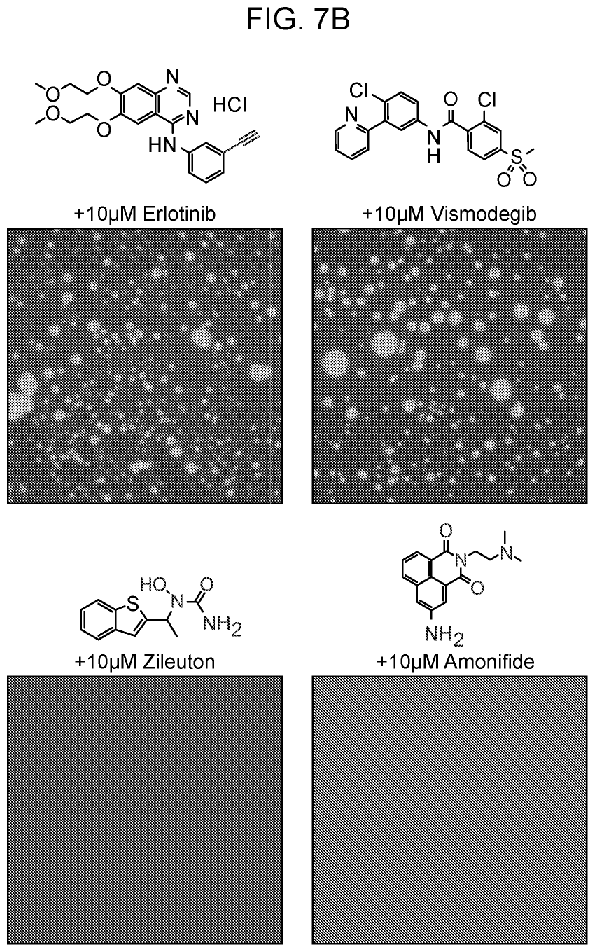

### In Vitro Droplet Assay (FIG. 3)

HP1α was diluted to the desired 2× concentration. HP1αKCK was added to HP1α at a ratio of 1:500 HP1α: HP1αKCK for evident fluorescent signal. HP1α-chromatin droplets were formed at 40 nM nucleosome array concentration in 20 mM HEPES pH 7.5, 0.1 mM EDTA and 2× HP1α-HP1αKCK in 20 mM HEPES pH 7.2, 150 mM KCl 1 mM DTT. 10 μL of 80 nM nucleosome arrays (2×) was mixed with 10 μl 2× HP1α-HP1αKCK and incubated at room temperature for 20 minutes before transferring to a glass bottom 384 well plate for imaging.

384 well glass bottom plates (Greiner Sensoplate 781892) were prepared for sample examination as follows: The wells were rinsed three times with 100 μl water, incubated with 100 μl of 2% Hellmanex for 30 mins-1 hour, rinsed with water three times again, incubated with 100 μl of 0.5M NaOH for 30 mins, rinsed with water three times, and then 70 μl of 20 mg/ml mPEG-silane dissolved in 95% EtOH was added to coat the wells. The plate was covered with foil and left overnight at 4° C.

After the overnight, the wells were rinsed with 95% EtOH five times, followed by incubation with 100 mg/ml BSA for 2 hours. Then, the wells were rinsed three times with water and three times with 1× phasing buffer afterwards (20 mM HEPES pH 7.2, 75 mM KCl, 0.05 mM EDTA, 0.5 mM DTT). After, the condensates are added to each well.

Screening with Small Molecules

To conduct droplet assays at scale, arrays assembled with 540 μg fluorescently labeled array DNA and 1.3 mg of fluorescently labeled HP1α were needed for one 384 well plate. 384 well plates were washed and coated in mPEG-silane in bulk using the Biotek EL406 washer dispenser. HP1α-array droplets were screened against a Bioactive compound library (Selleck Chemicals). These compounds were diluted in 1× phasing buffer (20 mM HEPES pH 7.2, 75 mM KCl, 0.05 mM EDTA, 0.5 mM DTT), and added to the wells for a final concentration of 10 μM in the reaction. The droplets and compounds were dispensed in the 384 wells using the Biomek-FX.

### Microscopy and Image Analysis

After 3-4 hours of settling to the bottom of the plate, the condensates were imaged on the IN Cell Analyzer 6500HS equipped with a Nikon Plan Apo 40×/0.95NA objective and sCMOS camera. Five field of views per channel were taken per well. Image analysis was conducted using a custom pipeline written in Python using NumPy and SciPy SciKit libraries. Classical image segmentation and quantification techniques were implemented to identify droplets and characterize their geometry, fluorescent intensity, and consequently the effectiveness of the small molecules on the droplets.

### Size Spectrum or Distribution Calculation

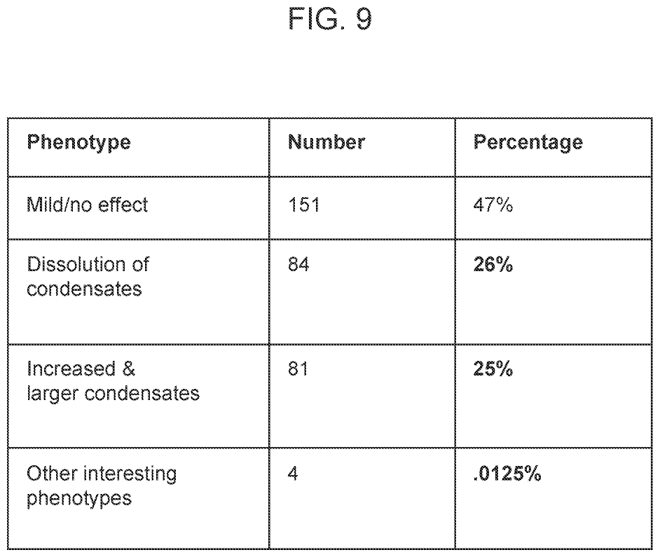

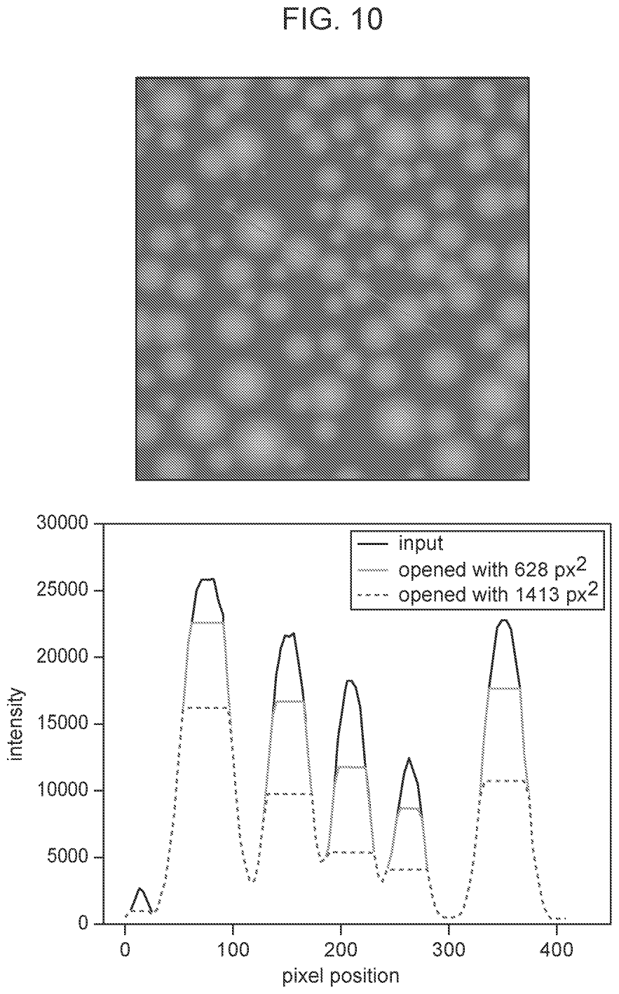

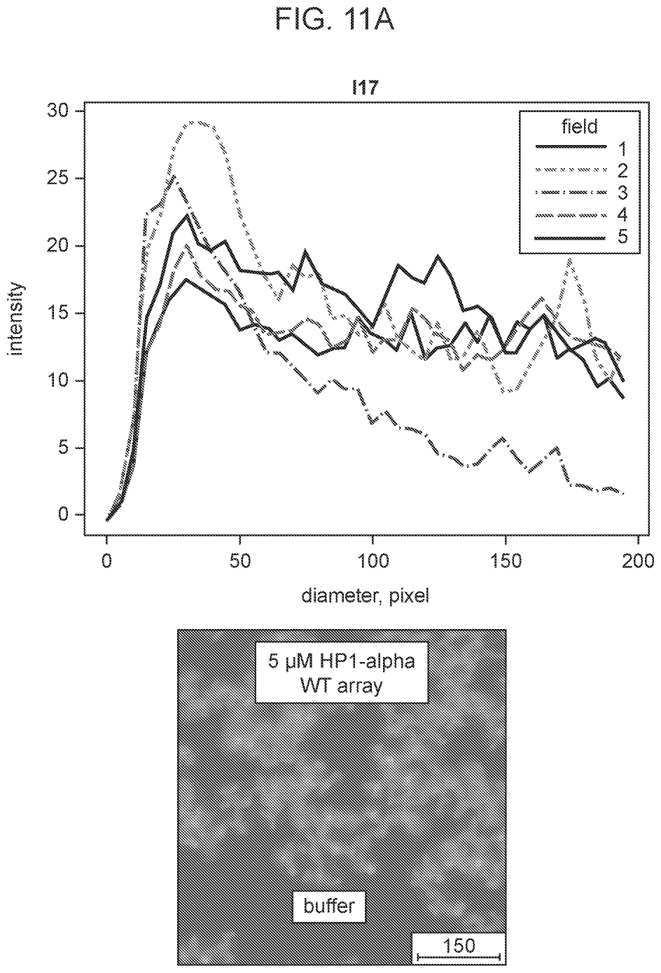

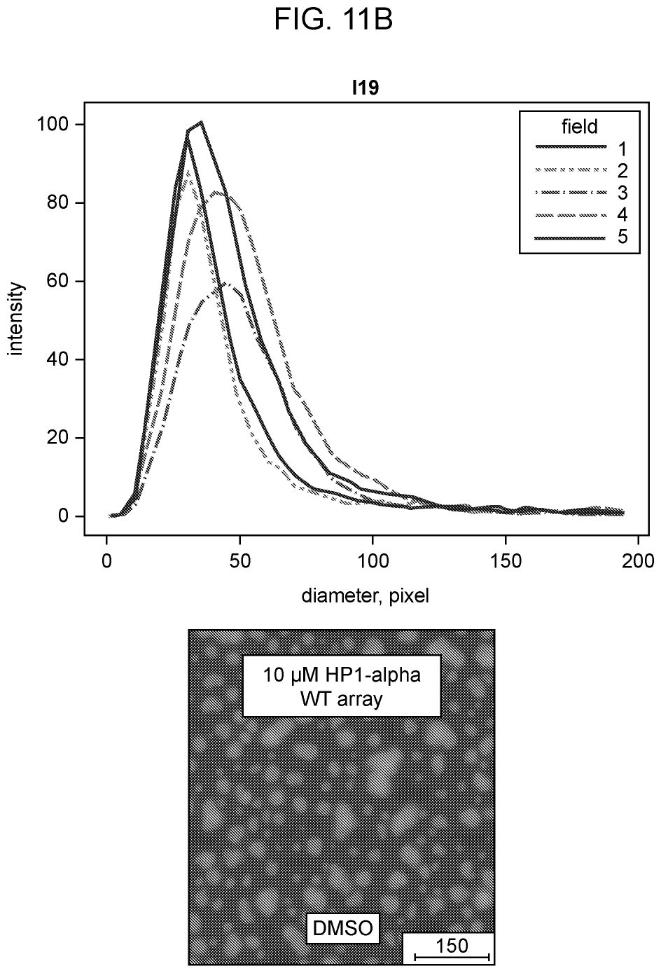

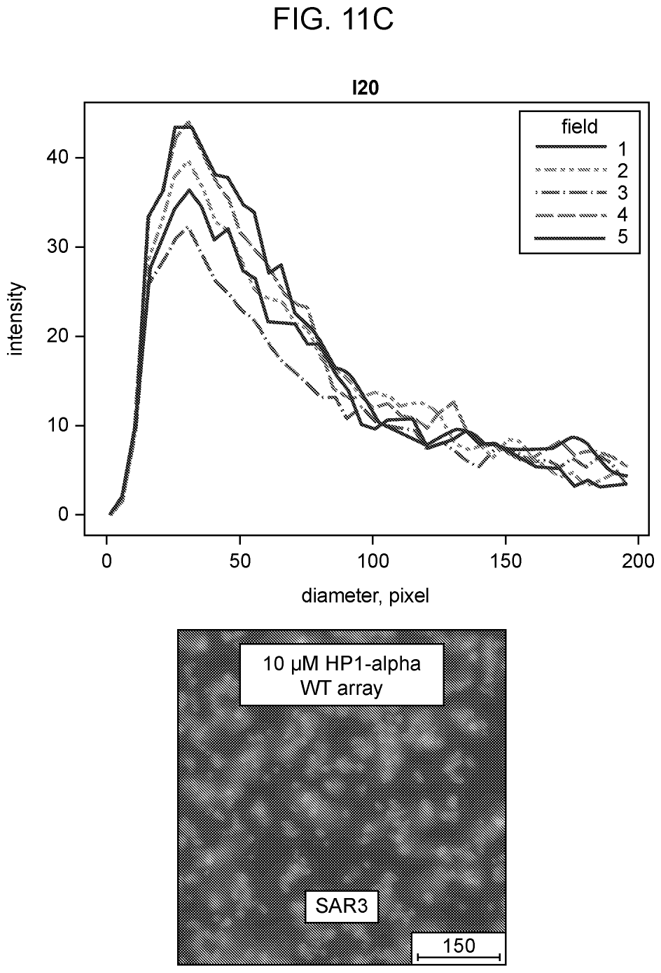

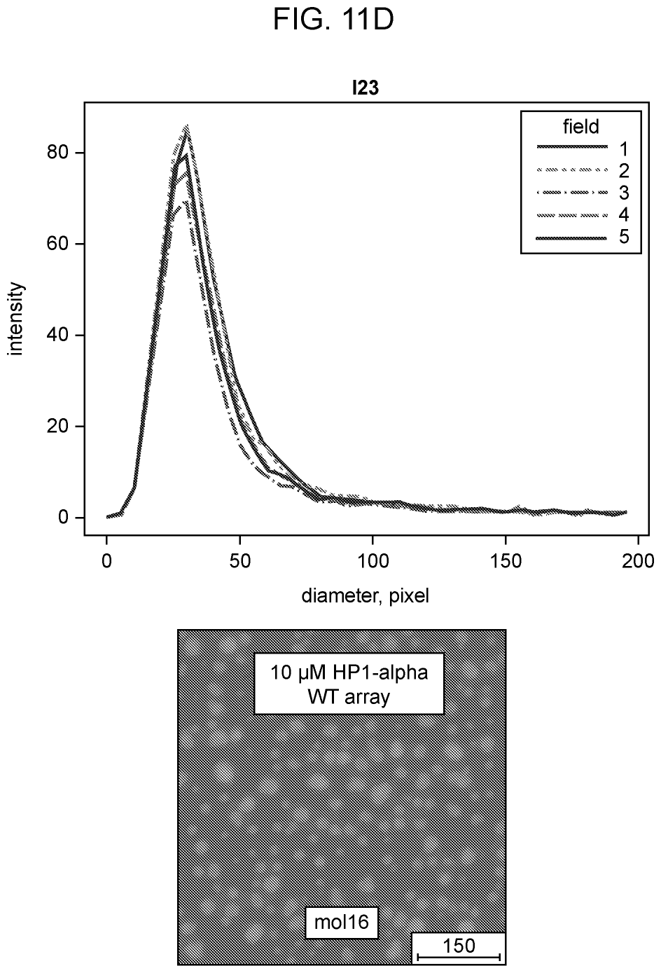

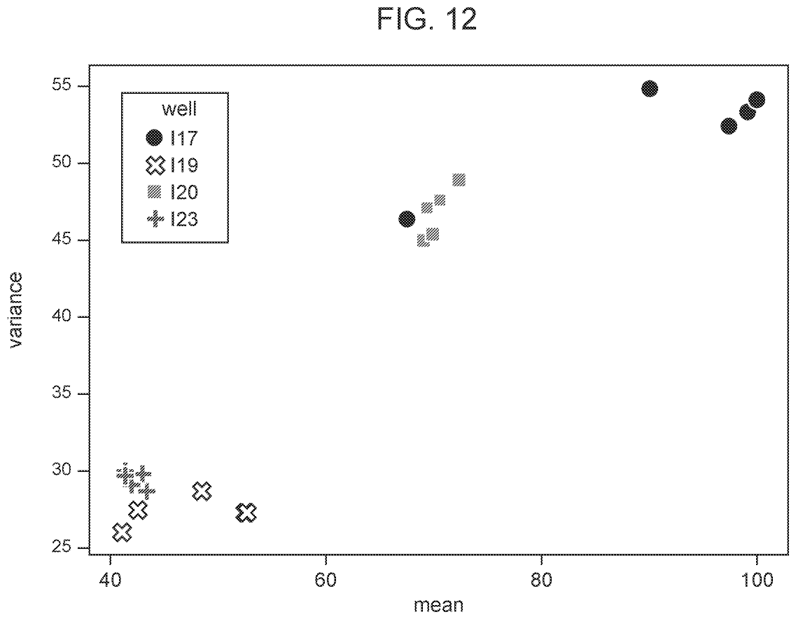

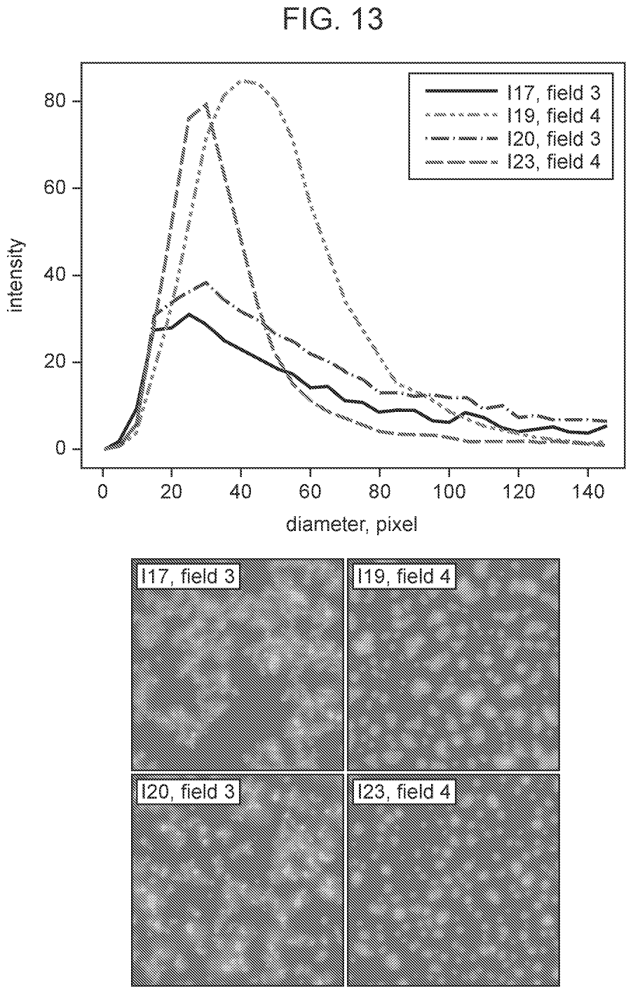

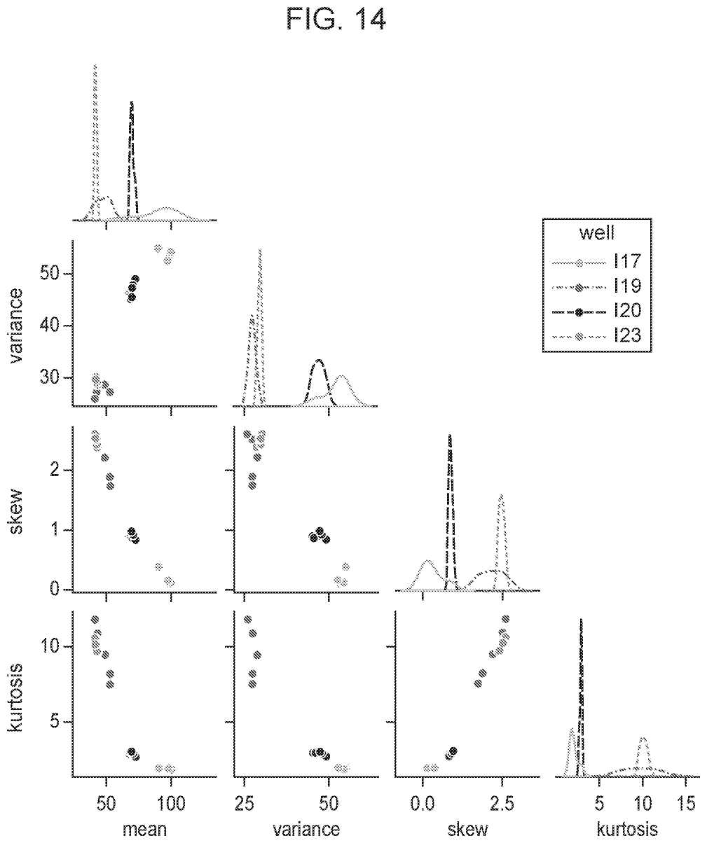

Images containing fluorescence signals of labeled chromatin arrays are pre-processed by resizing and their background signals removed. Since the visible features of interest are typically much larger than a single pixel, no information is lost and processing can be done faster on smaller images. The background signals typically only have low-frequency signal (slowly-varying) which is effectively removed by the “rolling-ball” algorithm (1). Size distribution is computed from the pre-processed images by successively opening (2) the input image with increasing area thresholds while keeping track of the total peak intensities lost due to area opening in each iteration (similar to method presented in ref. 3). No fixed footprint is used in opening the image and thresholding is not explicitly performed. Image opening produces “flat” peaks whose area equals the specified value and their peak heights lower than the original peak (FIG. 9). The surface areas used for opening the input image are calculated as areas of a circle with increasing diameter sizes. While in theory the process can be repeated until no intensities remain, stopping the algorithm to a specified maximum size is enough to obtain sufficient information and saves some computation time. To incorporate a global characteristic of the input image, the y-axis is scaled by the average intensity of the input image.

Size distributions are information-rich but are not practical in a high-throughput screen. To reduce the complexity of the size spectrum, standardized moments are computed from the size distributions. While no single number alone can sufficiently describe the entire size distribution, a few numbers comprising of higher-order moments (e.g. up to 4th-order, describing kurtosis of the distribution) can uniquely describe the size distribution. The equations for computing the moments are:

first moment (mean)

$$μ = \sum x * y * Δ x$$

second moment (variance)

$${σ}^{⋀} 2 = \sum {( x - μ )}^{⋀} 2 * y * Δ x$$

(standardized) third moment (skew)

$$\sum ( {( x - μ )}^{⋀} 3 / {σ}^{⋀} 3 ) * y * Δ x$$

(standardized) fourth moment (kurtosis)

$$\sum ( {( x - μ )}^{⋀} 4 / {σ}^{⋀} 4 ) * y * Δ x$$

Prior to computing these moments, y is normalized such that Σy*Δx=1. x is the diameter sizes used for opening. Area threshold is computed as 2π*(x/2)\hat{ }2. Δx is the spacing between diameter sizes.

### Example 2: Methods for Generating Chromatin States Associated with Particular Cancer Disease States

### 1. H3.3K27M Driven Pediatric Diffuse Intrinsic Pontine Glioma (DIPG)

These gliomas are driven by chromatin states that containing the human H3.3K27M mutation and are bound by the PRC2 complex and this aberrant interaction inhibits the normal action of the PRC2 complex (Schwartzentruber et al. Nature 2012).

To generate nucleosome array condensates that mimic this state, human histone H3.3 containing the H3.3K27M mutation was recombinantly expressed and purified from E. coli to make chromatin arrays bearing this onco-histone. The minimal PRC2 core complex, that is misregulated by the H3.3K27M histone (Lewis et al. Science 2013), was purified using previously described methods in insect cells (Grau et al. Nat. Comm. 2021). The PRC2 minimal complex was mixed with the arrays containing a precise mixture of H3.3K27M, H3.3 K27me3 and unmodified H3.3 to form condensates in vitro.

### 2. ETO1/AML1 and PML-RAR Fusion Protein Driven Acute Myeloid Leukemia (AML) and Acute Promyelocytic Leukemia (PML) Respectively.

These leukemias are driven by the oncogenic fusion proteins ETO1-AML1 and PML-RAR. Both types of fusion proteins recruit the HP1 heterochromatin machinery to genes that are normally developmentally regulated and cause constitutive repression thereby blocking normal myeloid differentiation (Minucci, S., et al., Carbone, R., et al., Reed-Inderbitzin, E., et al.).

To generate nucleosome array condensates that mimic the AML driving chromatin state, we will purify the ETO1-AML1 fusion protein using previously described methods (Klampfer et al. PNAS 1996). Methyl CpG binding protein 2 (MeCP2), which is recruited by ETO1-AML1 fusion protein (Fazi F et al. Blood 2007), will be purified using previously described methods (Li et al. Nature 2020). Native DNA CpG sequences will be cloned into plasmids, propagated an (d purified from E. coli cells using the above methods. The ETO1-AML1 fusion protein will then be mixed with MeCP2 and CpG methylated H3K9me3 heterochromatin arrays to form condensates in vitro. We will use analogous methods to purify PML-RAR and reconstitute nucleosome array condensates that mimic the PML driving chromatin state.

### 3. TEL/AML1 Fusion Protein Driven Pediatric Acute Lymphoblastic Leukemia (ALL)

These leukemias are driven by the oncogenic fusion protein TEL-AML1. The TEL-AML1 fusion protein recruits HP1 heterochromatin machinery to hematopoietic-specific genes, resulting in defects in hematopoietic development (Zelent et al. Oncogene 2004).

To generate chromatin condensates that mimic ALL resulting from the TEL-AML1 fusion protein, we will purify multiple components of the heterochromatin machinery involved in AML. Specifically, we will purify SUV39H1, a co-factor of AML (Durst et al. Oncogene 2004) and HP1 binding partner (Wang et al. Mol. Cell 2019), along with HP1 and TRIM28, which also binds to HP1 to promote condensate formation (Wang et al. Mol. Cell 2019). These proteins will be combined with the H3K9me3 chromatin array to form condensates in vitro.

### REFERENCES

Wu, M et al. Lysine-14 acetylation of histone H3 in chromatin confers resistance to the deacetylase and demethylase activities of an epigenetic silencing complex. eLIFE 7, e37231 (2018).
Luger, K et al. Preparation of nucleosome core particle from recombinant histones. Methods Enzymol. 304, 3-19 (1999).
Tsukiyama, T. Tsukiyama Lab Protocols. research.fredhutch.org/content/dam/stripe/tsukiyama/files/Protocols/expression.pdf.
Gibson, B A et al. Organization of chromatin by intrinsic and regulated phase separation. Cell 179, 470-484 (2019).
Sanulli, S et al. HP1 reshapes the nucleosome core to promote phase separation of heterochromatin. Nature 575, 390-394 (2019).
Schwartzentruber, J et al. Driver mutations in histone H3.3 and chromatin remodeling genes in pediatric glioblastoma. Nature 482, 226-231 (2012).
Lewis, P et al. Inhibition of PRC2 Activity by a Gain-of-Function H3 Mutation Found in Pediatric Glioblastoma. Science 340, 857-861 (2013).
Grau, D et al. Structures of monomeric and dimeric PRC2:EZH1 reveal flexible modules involved in chromatin compaction. Nat. Comm. 12, 741 (2021).
Klampfer, L et al. The AML1/ETO fusion protein activates transcription of BCL2. PNAS 93, 14059-14064 (1996).
Fazi, F et al. Heterochromatic gene repression of the retinoic acid pathway in acute myeloid leukemia. Blood 109, 4432-4440 (2007).
Li, C et al. MeCP2 links heterochromatin condensates and neurodevelopmental disease. Nature 586, 440-444 (2020).
Minucci, S., et al., Histone deacetylases: a common molecular target for differentiation treatment of acute myeloid leukemias?Oncogene, 2001. 20(24): p. 3110-5.
Carbone, R., et al., Recruitment of the histone methyltransferase SUV39H1 and its role in the oncogenic properties of the leukemia-associated PML-retinoic acid receptor fusion protein. Mol Cell Biol, 2006. 26(4): p. 1288-96.
Reed-Inderbitzin, E., et al., RUNX1 associates with histone deacetylases and SUV39H1 to repress transcription. Oncogene, 2006. 25(42): p. 5777-86.
Zelent, A et al. Role of the TEL-AML1 fusion gene in the molecular pathogenesis of childhood acute lymphoblastic leukemia. Oncogene 23, 4275-4283 (2004).
Durst, K et al. Role of RUNX family members in transcriptional repression and gene silencing. Oncogene 23, 4220-4224 (2004).
Wang, L et al. Histone modifications regulation chromatin compartmentalization by contributing to a phase separation mechanism. Mol. Cell 76, 646-659 (2019).
scikit-image.org/docs/stable/auto_examples/segmentation/plot_rolling_ball.html
scikit-image.org/docs/stable/api/skimage.morphology.html?highlight=area_opening #skimage.morphol ogy.area_opening
patents.justia.com/patent/20050186554—Method 3.3

### Example 3: Heterochromatin and Euchromatin Condensates

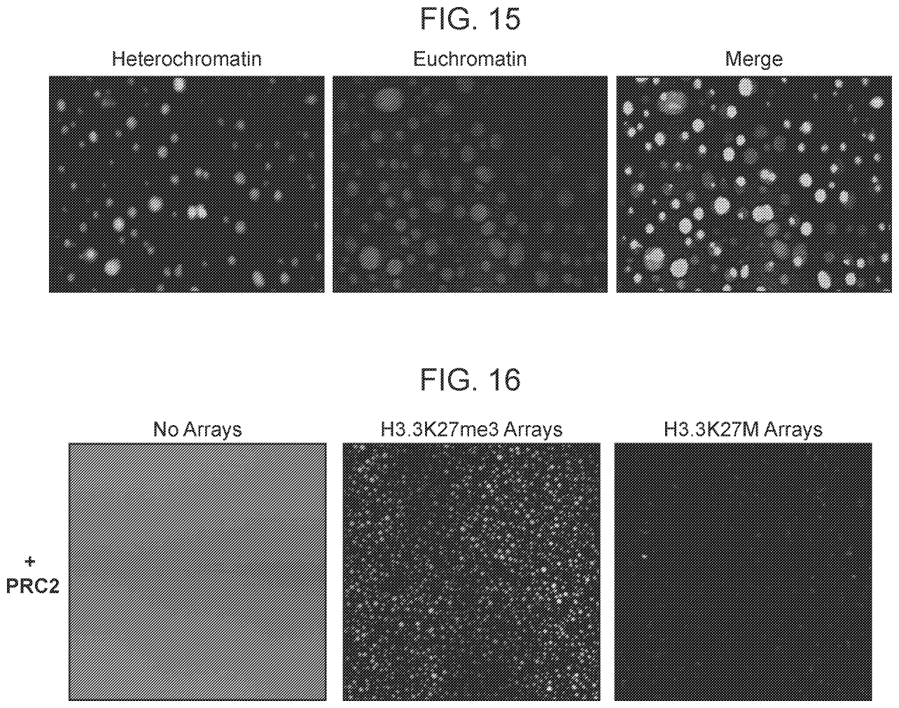

Previous studies have shown that both heterochromatin binding proteins and euchromatin binding proteins form condensates. However, the degree of specificity of euchromatin and heterochromatin condensates is unclear. Here it is shown for the first time that when HP1 H3K9me3 heterochromatin condensates are combined with Brd4 H3K27ac euchromatin condensates, the different types of chromatin condensates do not readily mix. In FIG. 15, fluorescently labeled HP1 heterochromatin droplets with H3K9me3 are shown in the upper left panel. Fluorescently labeled Brd4 euchromatin droplets with H3K27ac are shown in the upper right panel. As shown in the merged image (lower panel), the different types of chromatin condensates do not readily mix.

These results indicate that the biophysical and biochemical properties of the different types of chromatin condensates are distinct, and this difference will allow specific targeting of certain genome states.

### Example 4: The H3.3K27M Mutation Affects PRC2 Chromatin Condensate Formation

DIPG makes up about 75-80% of all pediatric brainstem cancers. The median survival range for DIPG is only 8-11 months after diagnosis because treatments are severely lacking. The defective genome organization in DIPG involves a specific mutation in one of the histone proteins, histone H3. The H3K27M mutation results in misregulation of PRC2 heterochromatin complex and globally decreases H3K27me3, which results in an aberrant heterochromatin state. The H3K27M mutation has been shown to inhibit PRC2 activity, but because the PRC2 binds to both H3K27 and H3K27M via similar mechanisms, it has been very difficult to identify drugs that can specifically overcome the H3K27M mutation.

Human PRC2 complex was added to both WT H3.3K27me3 nucleosome arrays and H3.3K27M nucleosome arrays. Droplets are visualized with end-labeled DNA. Human histones were purified as mentioned. H3.3K27M mutation was cloned into the pET3a vector for expression. The H3.3K27me3 was purified according to the methods. Arrays either consisted of 100% H3K27me3 octamer or 10% of H3K27M octamer and unmodified H3.3 octamer. The ratio of octamers used for the mutant array was chosen based on the amount of H3.3K27M found in DIPG patient chromatin. PRC2 complex was purified from insect cells using previously published methods as mentioned.

Here, it is demonstrated for the first time that the H3K27M mutation disrupts the ability of PRC2 to properly form condensates with chromatin. This difference will allow the specific identification of small molecules that can facilitate the restoration of the H3K27M-PRC2 condensate formation.

The amount of modified (H3K27me3) octamer and the amount of mutant (H3K27M) octamer can be titrated into the assembly to quantitatively incorporate the desired octamer into the chromatin array. For example, in FIG. 16, 10% of the H3K27M mutation was added in the chromatin (that is, 10% of the octamers were comprised of H3K27M histones), which reflects the amount of H3K27M incorporated into the genome of DIPG patients.

The above examples are provided to illustrate the invention but not to limit its scope. Other variants of the invention will be readily apparent to one of ordinary skill in the art and are encompassed by the appended claims. All publications, accessions, references, databases, and patents cited herein are hereby incorporated by reference for all purposes.

What is claimed is:

1. A method of assessing a test agent for the ability to modulate a chromatin state of interest, the method comprising:
contacting a phase-separated droplet comprising a nucleosome array condensate with the test agent; and
assessing the nucleosome array condensate for a modulation of the chromatin state of interest by the test compound.

2. The method according to claim 1, wherein the test agent is a small molecule, a biomolecule, or a polypeptide.

3. The method according to claim 1 or 2, wherein the chromatin state of interest mimics a naturally occurring chromatin state.

4. The method according to claim 3, wherein the naturally occurring chromatin state is a chromatin state of a normal cell.

5. The method according to claim 3, wherein the naturally occurring chromatin state is a chromatin state associated with a disease.

6. The method according to claim 5, wherein the disease is cancer, a neurodegenerative disease, or an autoimmune disease.

7. The method according to claim 6, wherein the chromatin state is specific to a type of cancer.

8. The method according to claim 7, wherein the cancer is diffuse intrinsic pontine glioma (DIPG).

9. The method according to any one of claims 1 to 8, wherein the nucleosome array condensate comprises histone modifications.

10. The method according to claim 9, wherein the histone modifications comprise one or more histone modifications selected from the group consisting of: H3K9me3, H4K20me3, H3K27me3, H3K4me3, H3K36me3, H3K79me3, H3K56ac, H3K9Ac, H4K16Ac, H4K14Ac, H4K18Ac, and any combination thereof.

11. The method according to claim 9 or claim 10, wherein the one or more histone modifications have a nucleosome occupancy selected to mimic a naturally occurring chromatin state.

12. The method according to claim 11, wherein the nucleosome occupancy is 60% or greater.

13. The method according to any one of claims 1 to 12, wherein the nucleosome array condensate comprises one or more histone variants.

14. The method according to claim 13, wherein the one or more histone variants comprise H2Az, macro H2A, H3.3, CENP-A, H2ABBD, or any combination thereof.

15. The method according to any one of claims 9 to 14, wherein the nucleosome array condensate comprises one or more heterochromatin or euchromatin proteins bound to the one or more histone modifications.

16. The method according to claim 15, wherein the one or more heterochromatin or euchromatin proteins bind cooperatively to the nucleosome array condensate.

17. The method according to claim 15 or 16, wherein the one or more heterochromatin or euchromatin proteins comprise chromobox protein homolog 5 (CBX5), chromobox protein homolog 3 (CBX3), chromobox protein homolog 1 (CBX1), polycomb repressive complex 1 (PRC1), polycomb repressive complex 2 (PRC2), or any combination thereof.

18. The method according to any one of claims 15 to 17, wherein the one or more heterochromatin or euchromatin proteins exhibit a specificity for histones of the nucleosome array of 2-fold or greater as compared to the specificity for histones of a corresponding nucleosome array in which the one or more histone modifications are absent.

19. The method according to any one of claims 15 to 18, wherein the one or more heterochromatin or euchromatin proteins exhibit a dissociation constant (K_{D}) for the nucleosome array of less than or equal to 5 μM.

20. The method according to any one of claims 15 to 19, wherein the one or more heterochromatin or euchromatin proteins are labeled, and wherein assessing the nucleosome array condensate for a modulation of the chromatin state of interest by the test compound comprises detecting the labeled heterochromatin or euchromatin proteins.

21. The method according to any one of claims 1 to 20, wherein the nucleosome array condensate comprises one or more proteins that associate with chromatin or DNA.

22. The method according to claim 21, wherein the one or more proteins that associate with chromatin or DNA comprise a polycomb-group protein, a bromodomain-containing protein, a chromobox protein homolog, a mediator complex subunit, or an oncogenic fusion protein, or any combination thereof.

23. The method according to claim 22, wherein the polycomb-group protein is PRC1 or PRC2, the bromodomain-containing protein is BRD1, BRD2, BRD3, BRD4, BRD5, BRD6, BRD7, BRD8, or BRD9, the chromobox protein homolog is CBX1, CBX2, CBX3, CBX4, CBX5, CBX6, CBX7, or CBX8, and the oncogenic fusion protein is AML-ETO.

24. The method according to any one of claims 1 to 23, wherein the nucleosome array condensate comprises labeled DNA, and wherein assessing the nucleosome array condensate for a modulation of the chromatin state of interest by the test compound comprises detecting the labeled DNA.

25. The method according to any one of claims 1 to 24, wherein the DNA used to assemble the nucleosome array is custom-designed to position nucleosomes at specific locations.

26. The method according to any one of claims 1 to 24, wherein the DNA used to assemble the nucleosome array comprises naturally occurring DNA sequences.

27. The method according to any one of claims 1 to 26, wherein the method is used to screen for and identify test agents that affect cooperativity or binding of proteins known to associate with chromatin or DNA or phase separation properties of chromatin.

28. The method according to any one of claims 1 to 27, further comprising counter-screening against other types of chromatin states to identify test agents that specifically modulate the chromatin state of interest.

29. The method according to any one of claims 1 to 28, wherein the method is a cell-free method.

30. The method according to any one of claims 1 to 29, wherein assessing the nucleosome array condensate for a modulation of the chromatin state of interest comprises assessing the nucleosome array condensate for a modulation of dissolution, shape change, size change, or any combination thereof.

31. A method to distinguish nucleosome array condensates that have different chromatin states, the method comprising:
g) performing fluorescence imaging of fluorescently labeled nucleosome array condensates to obtain fluorescence images;
h) performing segmentation-free image quantification on the fluorescence images;
i) computing size distributions of the nucleosome array condensates from the fluorescence images;
j) reducing complexities of the size distributions into distinct features that discriminate between the fluorescence images to simplify quantification of the nucleosome array condensates;
k) acquiring shape distributions for the nucleosome array condensates from the fluorescence images; and
l) reducing complexities of the shape distributions into distinct features that discriminate between the fluorescence images to simplify quantification of the nucleosome array condensates.

32. The method according to claim 31, further comprising quantifying z-factors using the outputs from steps d) and f) to assess quality control and reproducibility.

33. The method according to claim 31 or 32, further comprising comparing data from multiple screens to identify test agents that specifically modulate the chromatin state of interest.

34. A computer implemented method for processing fluorescence images of fluorescently labeled nucleosome array condensates, the computer performing steps comprising:
g) receiving the fluorescence images of the fluorescently labeled nucleosome array condensates;
h) performing segmentation-free image quantification on the fluorescence images;
i) computing size distributions of the nucleosome array condensates from the fluorescence images;
j) reducing complexities of the size distributions into distinct features that discriminate between the fluorescence images to simplify quantification of the nucleosome array condensates;
k) acquiring shape distributions for the nucleosome array condensates from the fluorescence images; and
l) reducing complexities of the shape distributions into distinct features that discriminate between the fluorescence images to simplify quantification of the nucleosome array condensates.

35. The method according to claim 34, further comprising quantifying z-factors using the outputs from steps d) and f) to assess quality control and reproducibility.

36. The method according to claim 34 or 35, further comprising comparing data from multiple screens to identify test agents that specifically modulate the chromatin state of interest.

37. A non-transitory computer-readable medium comprising program instructions that, when executed by a processor in a computer, causes the processor to perform the method of any one of claims 34 to 36.

38. A system for distinguishing nucleosome array condensates that have different chromatin states, the system comprising:
a processor programmed to process fluorescence images of fluorescently labeled nucleosome array condensates according to the computer implemented method of any one of claims 34 to 36; and
a display component for displaying information about the nucleosome array condensates regarding size distributions of the nucleosome array condensates, shape distributions of the nucleosome array condensates, quantification of the nucleosome array condensates, or histone modifications or DNA modifications, or any combination thereof.

39. The system according to claim 38, further comprising reagents for forming a phase-separated droplet comprising a fluorescently labeled nucleosome array condensate.

40. The system according to claim 38 or 39, further comprising a test agent.

41. A pharmaceutical composition comprising a test agent identified as modulating a chromatin state of interest by the method of any one of claims 1 to 28.

42. A method comprising administering to an individual in need thereof an effective amount of a test agent identified as modulating a chromatin state of interest by the method of any one of claims 1 to 28.

43. A method of producing a nucleosome array condensate that mimics a naturally occurring chromatin state, the method comprising: combining one or more heterochromatin or euchromatin proteins and a nucleosome array under conditions in which the nucleosome array forms a nucleosome array condensate that mimics a naturally occurring chromatin state, wherein the nucleosome array comprises histone modifications, histone variants, or both, selected to mimic the naturally occurring chromatin state.

44. The method according to claim 43, wherein the one or more heterochromatin or euchromatin proteins comprise CBX5, CBX3, CBX1, PRC1, PRC2, or any combination thereof.

45. The method according to claim 43 or claim 44, wherein the one or more heterochromatin or euchromatin proteins exhibit a specificity for histones of the nucleosome array of 2-fold or greater as compared to the specificity for histones of a corresponding nucleosome array in which the one or more histone modifications and/or histone variants are absent.

46. The method according to any one of claims 43 to 45, wherein the one or more heterochromatin or euchromatin proteins exhibit a dissociation constant (K_{D}) for the nucleosome array of less than or equal to 5 μM.

47. The method according to any one of claims 43 to 46, wherein the one or more heterochromatin or euchromatin proteins are labeled.

48. The method according to any one of claims 43 to 47, wherein the nucleosome array comprises histone modifications comprising H3K9me3, H4K20me3, H3K27me3, H3K4me3, H3K36me3, H3K79me3, H3K56ac, H3K9Ac, H4K16Ac, H4K14Ac, H4K18Ac, or any combination thereof, selected to mimic the naturally occurring chromatin state.

49. The method according to any one of claims 43 to 48, wherein the nucleosome array comprises histone variants comprising H2Az, macro H2A, H3.3, CENP-A, H2ABBD, or any combination thereof, selected to mimic the naturally occurring chromatin state.

50. The method according to any one of claims 43 to 49, further comprising adding one or more proteins that associate with chromatin or DNA to the nucleosome array condensate, wherein the one or more proteins that associate with chromatin or DNA comprise a polycomb-group protein, a bromodomain-containing protein, a chromobox protein homolog, a mediator complex subunit, or an oncogenic fusion protein, or any combination thereof, selected to mimic the naturally occurring chromatin state.

51. The method according to claim 50, wherein the polycomb-group protein is PRC1 or PRC2, the bromodomain-containing protein is BRD1, BRD2, BRD3, BRD4, BRD5, BRD6, BRD7, BRD8, or BRD9, the chromobox protein homolog is CBX1, CBX2, CBX3, CBX4, CBX5, CBX6, CBX7, or CBX8, and the oncogenic fusion protein is AML-ETO.

52. The method according to any one of claims 43 to 51, wherein the nucleosome array comprises labeled DNA.

53. The method according to any one of claims 43 to 52, wherein the method is a cell-free method.
